SEQUENCE LISTING (3 sequences)

SEQ ID NO: 1 - DNA, 10718 bp, Artificial
  Synthetic polynucleotide
agggggcggcgctcccggcccatcccttagccccgcggcggccgtgtgggccggaggctgcctgcaccgcgtcagggaggccggcctagaaaccctccctcccagaagaaagccgatcccagttcaggtggggtcttcctcggttgcgtacctggctggagccgagctggtgggcggccggcagccggcgtttctggtgatgacagccccgaaatgaaagcagcgcggccgccgcctccgagggctgcagggagatcagcgtccagcaaataagaagcaagtcctggacccggaggaggaggagcggccgagcatctctctctgctccgccgtgtcctttagatgagcactcccggccggagccggaggtggatccgcagagctgcctctgggcgcctgaccccgcgctgacatcacaacctgtgacaggcgcatcacgcccggtacctgctcccggccgctgcccgtcctcccagcctctttgtatgccgcagacatggccagccagcaggattcgggcttctttgagatcagtatcaaatatttactgaaatcctggagtaatacttctcccgttggcaacggttacatcaagcctccggttccacctgcttctggcacgcacagggagaaagggccgccaaccatgctacccatcaatgtggacccagacagtaaaccaggagaatatgtcctcaaaagtttatttgtcaacttcaccactcaggctgaacgcaagattcgtatcattatggcagagcccctggaaaagccattgacaaaatctctgcaacgtggagaagacccccaatttgatcaggtcatcagctcaatgagctccctttctgagtactgcctgccttccattctacgtacattatttgactggtataaaaggcaaaatggcattgaggatgaatcacatgaatacagaccaagaacaagcaataaatcaaaaagcgatgaacaacagcgagattatttaatggaaagacgggacctcgccattgattttattttttctttagtattaatagaagttttgaaacagattccacttcatcctgtaatagacagtttaatacatgatgttattaacttggctttcaagcactttaaatacaaagaagggtaccttggtcccaacactggcaatatgcatattgtggcagacctgtatgcagaagtcattggagtgttggcacaagccaaattccctgctgtaaagaagaaatttatggcggagctaaaagaattacggcacaaagagcagaacccatatgtggttcaaagcattatcagcttaataatgggcatgaaattctttcgaattaagatgtatccagtggaggattttgaggcctctcttcagtttatgcaggaatgtgcacattacttcctcgaggtcaaagacaaagatatcaagcatgccttggctgggctttttgttgaaatacttgttccagttgctgctgctgttaaaaatgaagtaaatgttccctgccttagaaattttgtggaaagcctgtatgacaccacgctggaactttcttctcgaaagaagcattccttggccttgtaccccctggtgacctgtttgctctgtgtcagtcagaagcagctgttcctgaacaggtggcacattttcctcaacaactgcttgtccaaccttaaaaacaaagatcccaagatggctcgagttgcactggaatctctctacagattactttgggtttacatgattcgaattaaatgtgaaagcaacacagctactcagagccgacttataaccatcatcacaacacttttccccaaagggtcccgcggtgtggtaccaagggacatgcctctgaacatctttgtgaaaatcatccagttcattgcccaggaacgtttagattttgcaatgaaagaaatcattttcgattttctttgtgtgggaaaaccagcaaaagcattcagtctcaacccagagagaatgaacattggtttacgggcattcttggtcatagctgatagcttgcagcagaaagatggggaacctcccatgccggttacaggagccgttcttccttcaggaaacacgttaagagtaaagaaaacatatttgagtaaaacactaactgaagaggaagccaaaatgataggcatgtccttatattactctcaagtacgaaaagctgtagacaacattttaaggcaccttgataaagaagtaggaaggtgtatgatgctgactaatgtacagatgttaaacaaagaaccggaagacatgatcacgggtgagagaaagccaaaaatagatcttttcaggacctgtgttgctgctattcctcgactgcttcctgatgggatgtcaaaacttgaacttattgacttactggctaggctctctattcatatggatgatgaattgcgacatattgcacaaaattctcttcagggtttacttgttgacttctcagattggagggaagatgtactatttggctttaccaacttcctgctccgggaagtaaatgatatgcatcacacactccttgattcgtccctgaagttgctgctgcagctgctcacccagtggaaactagtcatccagacacaaggaaaagtctatgaacaagccaacaaaatcagaaattcagagctcatcgcaaatggctccagtcacagaattcagtcggaacgaggtccccactgcagtgtactccacgctgtagaaggttttgctctggttttactctgcagtttccaggtggccacacgcaaactgtccgttttaatactcaaggaaattcgagcgttgtttattgccctggggcagcctgaggatgacgacaggccgatgattgatgtcatggatcagctaagttcttccattctagaaagttttattcatgtagcagtttcggattcagcaacattaccactcacccacaatgtggatctgcagtggttggtggaatggaacgcagtcctggtcaatagccattatgatgtgaaaagcccttcccatgtctggatatttgcacagtctgtcaaagacccctgggtcctctgcctcttcagcttcctccggcaggagaacttacccaagcactgccccacagccctcagctatgcctggccttatgccttcactcggctccagtcggtgatgcctctggtggacccaaatagcccaattaatgccaagaaaaccagcactgccggcagcggagacaactatgttactttgtggagaaattacctaattctttgttttggagttgcaaaacccagtattatgagcccaggacacttaagagcttccactccagaaataatggcgaccacacctgatggtacagtgagctacgataacaaggccataggcaccccatcggtgggagttctgttaaagcagttggtgcctttgatgagactagagagcattgagatcacagagtccttagttttaggatttggaagaacaaattcccttgttttcagagaattggtagaagaacttcatccattaatgaaagaagctctggaaagaagaccagagaacaagaaacgccgagaacggcgagacttgttaaggctacaactacttcgaatttttgaacttttggctgatgctggtgtaataagtgacagcacaaatggagccctagagcgggatactttagccctgggagctttgttcttagaatatgtggacttgacccgcatgctcctagaagctgaaaatgacaaagaagttgaaattcttaaagatatccgggcacattttagtgcaatggtggccaacttgattcagtgtgttccagttcaccaccgaagatttctcttcccccagcaaagtctgaggcaccaccttttcatcttattcagccagtgggcaggacccttcagcattatgttcactcctctggatcgttacagtgacagaaatcatcagattacaagatatcagtattgtgcattaaaagcaatgtcagcagtactgtgctgtggccctgtctttgacaatgtgggcctttccccagatggctacctatataaatggcttgacaacattctggcttgtcaagatttacgagttcatcaacttggctgcgaagttgttgtcttgctactggaacttaatcctgaccaaataaatctttttaactgggcaattgaccgatgctacacaggttcctaccaacttgcatctggctgcttcaaagccatagcaactgtgtgtggaagcaggaactatcccttcgacatagtgacattgttaaaccttgttctattcaaggcctctgacaccaacagagagatttatgaaatctccatgcagctcatgcagatccttgaagcaaagctttttgtatactcaaagaaagtcgctgagcaaagaccgggaagtattctctatggaacacacggcccgctgccacccctctacagcgtgtcacttgccctcttgtcatgtgagctggccaggatgtaccctgagctcacactccccctcttctcagaggtaagccagcgattccccacaacacaccccaacgggcgccagatcatgcttacctacctgctgccctggctgcacaacatcgagctggtggacagcaggctcctcctcccggggtcgagccccagcagcccagaggacgaagtcaaggaccgggaaggtgacgtgactgcttctcacgggctgagaggaaatggctggggctctccagaagccacgtcactggtcctgaacaacctcatgtacatgacggccaagtatggagatgaagttcctgggccagaaatggaaaatgcttggaatgctttagccaacaatgagaaatggagcaacaacctgaggatcaccttgcagttcctgattagcctctgtggggtcagcagcgacacagttctcctaccctatattaaaaaagtggcaatatacttgtgccgtaacaacaccattcaaaccatggaagagcttctctttgagctgcagcagacagagcccgtgaaccccatcgtccagcattgtgacaacccgcccttctaccgcttcacggccagtagcaaggcttccgcagcagcctcaggaaccacctctagcagcaatacagtggttgctggccaggaaaatttcccagatgctgaggagaacaagatattgaaagaatctgatgaaaggtttagtaatgtcatcagagcccacactcgcctcgagtcaagatacagcaatagctctggaggatcctacgatgaagataaaaatgatccaatttctccctacacgggctggttgctgactattacagagaccaagcagccgcagcccttaccgatgccttgtactggaggatgctgggcccccctggttgactatctcccggagaccatcactccccgggggccactccacaggtgcaatattgctgtaatttttatgactgaaatggtggtggatcacagtgtacgagaagactgggcgcttcatctaccattattacttcatgctgtcttcttaggtttagaccactaccggcctgaagtctttgaacacagcaaaaaactgcttcttcacctcttgattgccctctcttgcaacagcaatttccattccattgcttccgtgctcctgcagacccgagagatgggtgaagctaagactctaaccgtgcagccagcctaccaacctgaatatctctatacaggtggctttgacttcctgagagaggaccagtcatccccggtgcctgactcagggcttagttcaagctccacctcctctagcatcagtctgggaggcagcagtggaaacctcccacagatgacccaggaggtagaagatgtggacacagctgctgaaacagatgagaaggcaaacaagctcattgagtttctcacgaccagggcatttggtccactttggtgccatgaagacatcacacctaaaaatcaaaattcaaagagtgctgaacagctcactaattttctacgtcacgttgtatctgtatttaaagattccaaatcaggcttccatctggagcaccagttgagtgaagttgcattgcagacagccctcgcaagctcttcaaggcactatgctggtcggtccttccagatattccgggccctcaagcaacctctgtcagcacatgccttatctgaccttctctcaagattggtggaggtgataggagaacatggagatgagattcagggttatgtaatggaagcgctcctaaccttggaggcggctgtggataacttgtctgactgcttgaagaacagtgacctcctaactgtattgtcccgctcttcctcaccagatttaagctccagcagtaaactaacagcaagcagaaagagcacaggacaactaaacatgaacccgggaaccaccagcggcaacaccgcaactgccgaacggagccggcatcaacgaagcttctctgtgcccaagaagtttggtgtcatcgaccgatcctctgacccacctcgaagtgccacactggacagaattcaggcttgtacccaacaaggcctctcctcaaaaaccagaagctcatcctccttgaaggacagtctcacggacccatcccacataaaccatcccaccaacctgctggccaccatattctgggtcacagtggccttgatggagtctgattttgagtttgaatacttaatggccttaaggctgttgagcagactactggcacatatgccactcgataaggctgagaaccgagaaaagcttgagaaactccaggcacagctgaagtgggccgacttctccgggctgcagcagctgctgctgaaaggattcacatccctcaccaccacagacctgaccctgcagctcttcagtctgctgacaccagtgtccaaaatatccatggtggatgcatcccacgctattgggtttccactgaatgtcttgtgtctcctgcctcagctgattcagcattttgaaaatcccaatcagttctgtaaggatatagccgaaaggattgctcaggtttgtttagaagagaagaaccccaaactttcaaatcttgcacatgtcatgactctttataaaacgcacagctacacgagggactgtgccacgtgggtcaatgtggtctgtcgataccttcatgaagcatatgctgacattaccttgaatatggttacctacctggcagagctgctggagaagggcctccctagtgtgcagcagcccctgctccaggtgatctacagtcttctcagctacatggacctttctgtcgttcctgtcaaacagttcaatgtggaagttctgaagacaattgaaaaatatgtgcaaagtgttcactggagagaagctctgaatatcttgaagctggtagtttctcggtcagccagccttgttttaccttcataccagcacagtgacctctcaaaaatagaaatacatcgagtgtggactagtgcttccaaggaattacctgggaaaaccctggacttccacttcgatatttcggagactccaatcatcgggaggcggtatgatgagctgcagaattcttctgggcgtgatgggaagcccagggccatggccgtcacccggagcacatcttccacttcctcaggctccaactccaacgtccttgttccagtgagctggaaaaggccccagtattctcagaagagaacaaaagagaagttggtacatgtcctttctctgtgtggccaagaagtaggattgagcaaaaatccatcagtgattttttcatcgtgtggggatctggatctgcttgagcaccagacaagcttggtatcttctgaggacggtgcccgagagcaggagaacatggatgacacaaacagcgagcagcagtttagagtcttcagagacttcgacttcctagatgtggagctggaggatggagagggtgagagtatggacaatttcaactggggagtgcgcagacgttctctggacagcctggataagtgtgatatgcagattctggaggagcgccaactgtcaggaagcactcctagcctgaataaaatgcaccatgaggactccgatgaatcatccgaggaggaggacctcacagccagccagatcctggagcactcagacctaatcatgactctctccccctctgaagagacgaatcccatggagctgctcaccacagcctgtgactcgacccctgcagaacctcattcctttaacaccagaatgtccagctttgatgcttccttgcctgatatgaataatctgcagatttctgagggttcaaaggctgaagctgttcgtgaggaggaggacaccaccgtgcatgaggatgatctttctagttccatcaatgaactcccagcagcttttgaatgcagcgacagctttagcctggacatgactgagggggaagaaaaaggcaatcgggcactggaccagtttaccctggcgagctttggagaaggtgacaggggagtctctccccctccctcgcccttcttctcagccatccttgccgcctttcagcccgcagcctgtgacgatgccgaggaggcctggcgcagccacatcaaccagcttatgtgtgactcagatggctcctgtgctgtgtatacatttcatgtgttctcctccttgtttaagaatattcagaaaaggttctgcttcctaacctgtgatgcagccagttaccttggagataacctccggggaatcggatccaaatttgtcagctcttcccagatgctcacctcctgctctgaatgtcctacactttttgtggatgccgagactctcctttcatgtggacttctggacaagctcaagttcagtgtgttagaactgcaagaatatttggatacctacaacaacaggaaagaggccacactctcttggcttgcaaattgtaaggcaacatttgcagggggatcaagagatggagtaattacctgtcaaccaggggactccgaagaaaagcaattggaactgtgtcagagattatataagctacacttccagctgctattgctttttcagtcctactgtaagctcatcggccaggtgcacgaagttagctccatgccagagctgctgaatatgtccagggaactgagtgacctaaagaaacacctgaaggaagccagtgcagtcattgcagctgaccctctctattcagacggcgcgtggtccgagcccaccttcacgtccactgaagcagccatccagtccatgctggagtgcctgaagaacaacgaactcggcaaagctttgcggcagatcagggagtgcagaagtctgtggcccaatgacatctttggaagcagttctgatgatgaggtccagacactactgaatatttatttccgtcaccaaactctgggacagacgggtacttatgccctggtggggtctaaccagagcctgaccgagatctgcaccaagctgatggagctgaacatggagatccgggacatgatccgcagggcccagagttaccgagtcctcactacttttcttccagactccagtgtttctggcactagtctctgacaggagcctcctgtccccactgggttccaaactggcagtgctgccatgctggggcaacgtcattcagtgtcttctcggccttcaaaaggcttggacagactgttctccctcttgttacctgtagggctttttctaaagaggatggcagaacttccaacgtgtagcaatactataagaaccaaggtagcttagaacgtcctggacagactccactcatcatgctgtgtggcacaaatgtgttacatttgaccgagcatatgcaactcgctactgaagaagtgacttccgttgcataccaaagccgactacactgaacagtaccttcctttctagaaacaattttagattggcaaaagtgcaatgttttcttcactcaaaaaattttatattctcaaacatgtatattctttccctgtcttgttccattttcttttcttttttcttttttctttttcctttctttcgtgggctgagaaaggggcaggcaaaatgaagctggccactgaaaactgtaagatggtcaaaagctgacagcctgtgtatgtgaaaagggaattgtaaatggactgcaatgtaatgtacactgtaatttgaatacaattactgtatctaaaaggagctgctatgaagtacctttcttatgttgctaggctactgtttctgaaagccctggatctctttgcaccaaaaatggtccagatagactctttttaaggatcttggctgctttttactagaaggttgcttttatgagcatatttatactgctgaaggatgagtgttaattttaattaactttgccgttttgtagagaaaactattcacaagataaattccaagtcttttcacctgtcaggcatgcatattttaatatctgtttggatagtcagaagtagaatcataaaggtaaaatatgagttgttactttgtttcttcgatgtcatattttatgtgtaatatatatgtaaagggccattcttaagttctctccttaaacttaatgctgtcaagtgttagatgtgtgcatgtgaacttgttgcactgcagaaacatattcagagtttatctatgtaacttattcactctgtaaatacatttaaagtttttgtgatgtaagcttaattgatattctgttcagaactttctttagactaaaaaaaaaaaaaaagacaaaatacaa

SEQ ID NO: 2 - protein, 3013 aa, Artificial
  Synthetic polypeptide
MASQQDSGFFEISIKYLLKSWSNTSPVGNGYIKPPVPPASGTHREKGPPTMLPINVDPDSKPGEYVLKSLFVNFTTQAERKIRIIMAEPLEKPLTKSLQRGEDPQFDQVISSMSSLSEYCLPSILRTLFDWYKRQNGIEDESHEYRPRTSNKSKSDEQQRDYLMERRDLAIDFIFSLVLIEVLKQIPLHPVIDSLIHDVINLAFKHFKYKEGYLGPNTGNMHIVADLYAEVIGVLAQAKFPAVKKKFMAELKELRHKEQNPYVVQSIISLIMGMKFFRIKMYPVEDFEASLQFMQECAHYFLEVKDKDIKHALAGLFVEILVPVAAAVKNEVNVPCLRNFVESLYDTTLELSSRKKHSLALYPLVTCLLCVSQKQLFLNRWHIFLNNCLSNLKNKDPKMARVALESLYRLLWVYMIRIKCESNTATQSRLITIITTLFPKGSRGVVPRDMPLNIFVKIIQFIAQERLDFAMKEIIFDFLCVGKPAKAFSLNPERMNIGLRAFLVIADSLQQKDGEPPMPVTGAVLPSGNTLRVKKTYLSKTLTEEEAKMIGMSLYYSQVRKAVDNILRHLDKEVGRCMMLTNVQMLNKEPEDMITGERKPKIDLFRTCVAAIPRLLPDGMSKLELIDLLARLSIHMDDELRHIAQNSLQGLLVDFSDWREDVLFGFTNFLLREVNDMHHTLLDSSLKLLLQLLTQWKLVIQTQGKVYEQANKIRNSELIANGSSHRIQSERGPHCSVLHAVEGFALVLLCSFQVATRKLSVLILKEIRALFIALGQPEDDDRPMIDVMDQLSSSILESFIHVAVSDSATLPLTHNVDLQWLVEWNAVLVNSHYDVKSPSHVWIFAQSVKDPWVLCLFSFLRQENLPKHCPTALSYAWPYAFTRLQSVMPLVDPNSPINAKKTSTAGSGDNYVTLWRNYLILCFGVAKPSIMSPGHLRASTPEIMATTPDGTVSYDNKAIGTPSVGVLLKQLVPLMRLESIEITESLVLGFGRTNSLVFRELVEELHPLMKEALERRPENKKRRERRDLLRLQLLRIFELLADAGVISDSTNGALERDTLALGALFLEYVDLTRMLLEAENDKEVEILKDIRAHFSAMVANLIQCVPVHHRRFLFPQQSLRHHLFILFSQWAGPFSIMFTPLDRYSDRNHQITRYQYCALKAMSAVLCCGPVFDNVGLSPDGYLYKWLDNILACQDLRVHQLGCEVVVLLLELNPDQINLFNWAIDRCYTGSYQLASGCFKAIATVCGSRNYPFDIVTLLNLVLFKASDTNREIYEISMQLMQILEAKLFVYSKKVAEQRPGSILYGTHGPLPPLYSVSLALLSCELARMYPELTLPLFSEVSQRFPTTHPNGRQIMLTYLLPWLHNIELVDSRLLLPGSSPSSPEDEVKDREGDVTASHGLRGNGWGSPEATSLVLNNLMYMTAKYGDEVPGPEMENAWNALANNEKWSNNLRITLQFLISLCGVSSDTVLLPYIKKVAIYLCRNNTIQTMEELLFELQQTEPVNPIVQHCDNPPFYRFTASSKASAAASGTTSSSNTVVAGQENFPDAEENKILKESDERFSNVIRAHTRLESRYSNSSGGSYDEDKNDPISPYTGWLLTITETKQPQPLPMPCTGGCWAPLVDYLPETITPRGPLHRCNIAVIFMTEMVVDHSVREDWALHLPLLLHAVFLGLDHYRPEVFEHSKKLLLHLLIALSCNSNFHSIASVLLQTREMGEAKTLTVQPAYQPEYLYTGGFDFLREDQSSPVPDSGLSSSSTSSSISLGGSSGNLPQMTQEVEDVDTAAETDEKANKLIEFLTTRAFGPLWCHEDITPKNQNSKSAEQLTNFLRHVVSVFKDSKSGFHLEHQLSEVALQTALASSSRHYAGRSFQIFRALKQPLSAHALSDLLSRLVEVIGEHGDEIQGYVMEALLTLEAAVDNLSDCLKNSDLLTVLSRSSSPDLSSSSKLTASRKSTGQLNMNPGTTSGNTATAERSRHQRSFSVPKKFGVIDRSSDPPRSATLDRIQACTQQGLSSKTRSSSSLKDSLTDPSHINHPTNLLATIFWVTVALMESDFEFEYLMALRLLSRLLAHMPLDKAENREKLEKLQAQLKWADFSGLQQLLLKGFTSLTTTDLTLQLFSLLTPVSKISMVDASHAIGFPLNVLCLLPQLIQHFENPNQFCKDIAERIAQVCLEEKNPKLSNLAHVMTLYKTHSYTRDCATWVNVVCRYLHEAYADITLNMVTYLAELLEKGLPSVQQPLLQVIYSLLSYMDLSVVPVKQFNVEVLKTIEKYVQSVHWREALNILKLVVSRSASLVLPSYQHSDLSKIEIHRVWTSASKELPGKTLDFHFDISETPIIGRRYDELQNSSGRDGKPRAMAVTRSTSSTSSGSNSNVLVPVSWKRPQYSQKRTKEKLVHVLSLCGQEVGLSKNPSVIFSSCGDLDLLEHQTSLVSSEDGAREQENMDDTNSEQQFRVFRDFDFLDVELEDGEGESMDNFNWGVRRRSLDSLDKCDMQILEERQLSGSTPSLNKMHHEDSDESSEEEDLTASQILEHSDLIMTLSPSEETNPMELLTTACDSTPAEPHSFNTRMSSFDASLPDMNNLQISEGSKAEAVREEEDTTVHEDDLSSSINELPAAFECSDSFSLDMTEGEEKGNRALDQFTLASFGEGDRGVSPPPSPFFSAILAAFQPAACDDAEEAWRSHINQLMCDSDGSCAVYTFHVFSSLFKNIQKRFCFLTCDAASYLGDNLRGIGSKFVSSSQMLTSCSECPTLFVDAETLLSCGLLDKLKFSVLELQEYLDTYNNRKEATLSWLANCKATFAGGSRDGVITCQPGDSEEKQLELCQRLYKLHFQLLLLFQSYCKLIGQVHEVSSMPELLNMSRELSDLKKHLKEASAVIAADPLYSDGAWSEPTFTSTEAAIQSMLECLKNNELGKALRQIRECRSLWPNDIFGSSSDDEVQTLLNIYFRHQTLGQTGTYALVGSNQSLTEICTKLMELNMEIRDMIRRAQSYRVLTTFLPDSSVSGTSL

SEQ ID NO: 3 - protein, 14 aa, Homo sapiens
WGVRRRSLDSLDKC

(19) United States
(12) Patent Application Publication
(10) Pub. No.: US 2014/0295416 A1
(43) Pub. Date: Oct. 2, 2014

(54) Novel Method of Cancer Diagnosis and Prognosis and Prediction of Response to Therapy

(71) Applicants: Helmut Zarbl, Princeton, NJ (US); Jessica Graham, Plainsboro, NJ (US)

(72) Inventors: Helmut Zarbl, Princeton, NJ (US); Jessica Graham, Plainsboro, NJ (US)

(73) Assignee: University of Medicine and Dentistry of New Jersey, Somerset, NJ (US)

(21) Appl. No.: 13/992,134

(22) PCT Filed: Dec. 6, 2011
(86) PCT No.: PCT/US11/63553

§ 371 Date: Mar. 7, 2014

Related U.S. Application Data
(60) Provisional application No. 61/419,975, filed on Dec. 6, 2010.

(51) Int. Cl. C12Q 1/68; G01N 33/574; C07K 14/47

(52) U.S. Cl. CPC C12Q 1/6886; C07K 14/47; G01N 33/57496
USPC 435/6.11; 530/387.9; 435/7.1

(57) ABSTRACT

The invention relates to pharmaceutical compositions comprising of FRY polypeptides and nucleotides, methods to treat cancer, methods to diagnose cancer, and methods to determine the effectiveness of the treatment of cancer, as well as methods to differentiate stem cells.

### CROSS REFERENCE TO RELATED APPLICATION

This application claims priority of U.S. Provisional Application No. 61/419,975 filed on Dec. 6, 2010. The content of the application is incorporated herein by reference in its entirety.

### GOVERNMENT SUPPORT

The work described herein was funded, in whole or in part, by grant number NCI CA 77222 from the National Institutes of Health. The United States Government has certain rights in the invention.

### FIELD OF THE INVENTION

The invention relates to pharmaceutical compositions comprising FRY polypeptides and polynucleotides, methods to treat cancer, methods for the diagnosis and prognosis of cancer, and methods to determine the effectiveness of the treatment of cancer, as well as methods to differentiate stem cells.

### BACKGROUND OF THE INVENTION

Breast cancer remains the most prevalent cancer among US women, with an estimated 207,090 new cases and 39,840 deaths occurring in 2010 (National Cancer Institute SEER Cancer Statistics). Although numerous genetic alterations and molecular pathways are implicated in the pathogenesis of breast cancer, significant gaps remain in our knowledge of genetic susceptibility. Genetic linkage studies in high-risk families identified BRCA1 and BRCA2; however, these breast cancer susceptibility genes are implicated in less than 10% of all cancers and account for only a fraction of familial breast cancers. Subsequent family-based studies identified a putative suppressor (BRCA3) distal to BRCA2 on human chromosome 13. However, the latter was not confirmed in additional cohorts, attesting to the difficulty of linkage analysis, even in high-risk cancer families.

As an alternative approach to identifying cancer susceptibility genes, researchers have performed linkage analysis in inbred animal models of heritable cancer. For example, genetic studies in differentially susceptible rat strains have identified more than twenty mammary carcinoma susceptibility (Mcs) loci, although the putative suppressor genes within these loci have yet to be identified and validated. Previous segregation analyses indicated that a cross between the resistant Copenhagen (Cop) and the intermediately-sensitive Fisher 344 (F344) strains yielded a minimal number of genetic modifiers.

A current need exists for the identification of additional predictive markers for the diagnosis and prognosis of breast cancer and other cancers arising from epithelial cells.

### SUMMARY OF THE INVENTION

This invention generally relates to pharmaceutical compositions that contain FRY polypeptides or FRY polynucleotides, as well as methods of treating cancer, methods of diagnosing cancer, methods of determining cancer phenotype, methods to determine the effectiveness of the treatment of cancer and methods to differentiate stem cells, as well as kits for performing the methods of the invention.

In one aspect, the invention provides pharmaceutical compositions comprising a FRY polypeptide or FRY polynucleotides that encode a FRY polypeptide and a pharmaceutically acceptable carrier. In certain embodiments, the FRY polypeptide is a recombinant polypeptide, and may be a fusion polypeptide. In other embodiments, the FRY polynucleotides are contained in a vector. Such vectors include expression vectors, as well as viral vectors capable of delivering the FRY polynucleotide to cancer cells.

In a second aspect, the invention provides a method for treating cancer comprising administering to a subject in need thereof an effective amount of a pharmaceutical composition comprising a FRY polypeptide or a FRY polynucleotide encoding a FRY polypeptide. In certain embodiments, the methods comprise reducing the number of cancer cells in a tumor, reducing the size of the tumor, enhancing the efficacy of chemotherapeutics and/or reducing the spread of cancer to peripheral organs.

The types of cancers that may be treated using the pharmaceutical compositions described herein include epithelial cell cancer, breast cancer, prostate cancer, ovarian cancer, lung cancer, brain cancer and blood cancer. The cancer cells generally contain a low level of the FRY polypeptide and FRY mRNA compared to normal cells from the same type of non-cancerous tissue, or lack a functional FRY polypeptide or gene. The cancer cells generally possess a stem cell phenotype. The cancer may be breast cancer, a hormone receptor negative breast cancer, or a triple negative breast cancer, in which the breast cancer cells lack the estrogen receptor (ER), the progesterone receptor (PR) and the human epidermal growth factor receptor 2 (Her2).

In certain embodiments, the FRY polynucleotide encoding a FRY polypeptide is delivered to a cancer cell using a vector. In certain embodiments, the vector is an expression vector, or a viral vector capable of delivering the FRY polynucleotide to a cancer cell. The vector includes a nucleic acid in a form suitable for expression of the nucleic acid in a cancer cell. Preferably the vector includes one or more regulatory sequences operatively linked to the nucleic acid sequence to be expressed. The term regulatory sequence includes promoters, enhancers, and other expression control elements (e.g., splicing signals, untranslated regions, polyadenylation signals). Regulatory sequences include those that direct constitutive expression of a nucleotide sequence, as well as tissue-specific regulatory and/or inducible sequences. The design of the expression vector can depend on such factors as the choice of the cancer cell to be transformed, the level of transcription of RNA desired, and the like. In certain embodiments, the viral vector is an adenovirus vector, a lentivirus vector, an adeno-associated virus vector, a pox virus vector, an alphavirus vector, or a herpes virus vector.

In a third aspect, the invention provides an isolated antibody that specifically binds to a FRY polypeptide. In certain embodiments the antibody binds to an epitope comprising WGVRRRSLDSLDKC (SEQ ID NO. 3) within the FRY polypeptide.

In a fourth aspect, the invention provides methods for determining the effectiveness of a treatment in a subject suffering from cancer comprising obtaining a pretreatment sample that contains cancer cells from the subject; obtaining a post treatment sample that contains cancer cells; detecting the level of the FRY polypeptide present in the samples; comparing the level of the FRY polypeptide in the pretreatment sample to the post treatment sample; wherein the treatment is determined to be effective if the FRY polypeptide level present in the post treatment sample is increased compared to the FRY polypeptide level present in the pretreatment sample. In certain embodiments, the FRY polypeptide in a cancer cell can be detected using an antibody that binds to the FRY polypeptide. In certain embodiments, the FRY antibody is an antibody that binds to WGVRRRSLDSLDKC (SEQ ID NO. 3).

In a fifth aspect, the invention provides methods for diagnosing cancer in a subject comprising detecting a level of a FRY polypeptide or a polynucleotide encoding a FRY polypeptide in a sample from the subject; and comparing the level detected in the subject's sample to the level of a FRY polypeptide expressed in a normal cell from the same type of non-cancerous tissue. In certain embodiments, the FRY polypeptide in a cancer cell is detected using an antibody that binds to the FRY polypeptide. In certain embodiments, the FRY antibody is an antibody that binds to WGVRRRSLDSLDKC (SEQ ID NO. 3).

In a sixth aspect, the invention provides methods to assess the prognosis of a cancer in a subject comprising detecting a level of a FRY polypeptide or a polynucleotide encoding a FRY polypeptide in a biological sample from the subject; and comparing the level detected in the subject's sample to a cancer grading system wherein the prognosis of the cancer is determined according to the level of the FRY polypeptide and the grade of the cancer. In certain embodiments, the FRY polypeptide in a cancer cell can be detected using an antibody that binds to the FRY polypeptide. In certain embodiments, the FRY antibody is an antibody that binds to WGVRRRSLDSLDKC (SEQ ID NO. 3).

The types of cancers that may be diagnosed and for which progress can be assessed and treatment effectiveness determined using the methods described herein include epithelial cell cancer, breast cancer, prostate cancer, ovarian cancer, lung cancer, brain cancer and blood cancer. The cancer cells generally contain a low level of the FRY polypeptide and FRY mRNA compared to normal cells from the same type of non-cancerous tissue, or lack a functional FRY polypeptide or gene. The cancer cells generally possess a stem cell phenotype. The cancer may be breast cancer, a hormone receptor negative breast cancer, or a triple negative breast cancer, in which the breast cancer cells lack the estrogen receptor (ER), the progesterone receptor (PR) and the human epidermal growth factor receptor 2 (Her2).

In a seventh aspect, the invention provides for methods of screening for candidate compounds that increase the expression or activity of the FRY gene in a cell; measuring the expression level of the FRY gene or activity of the FRY polypeptide of said contacted cells; wherein an increase of the level of the FRY polypeptide identifies said candidate agent as a compound to increase expression or activity of the FRY gene compared to untreated cells. In certain embodiments, the cells are cancer cells, cancer cells that express a low level of the FRY polypeptide, cancer cells that possess a stem cell phenotype, as well as breast cancer cells.

In an eighth aspect, the invention provides for methods to differentiate a cell that expresses a low level of FRY polypeptide comprising introducing into the cell a polynucleotide encoding a FRY polypeptide or a FRY polypeptide.

In a ninth aspect, the invention provides kits for performing the methods of the invention.

### BRIEF DESCRIPTION OF THE DRAWINGS

FIGS. 1a-b depicts the comparative protein sequence alignment of FRY gene homologs from different species: FIG. 1 (a) The nonsynonymous SNP in the F344 rat FRY allele at codon 661 substitutes an Aspartic acid (D) residue in Cop protein with Glutamic acid (E) in the F344 protein. Notice that the Aspartic acid residue is highly conserved among species; FIG. 1 (b) The nonsynonymous SNP in the F344 rat FRY allele at codon 2170 substitutes a nonpolar Alanine (A) in the Cop protein with a polar Serine (S) residue in F344 protein. Notice that the Alanine residue is highly conserved among species.

FIGS. 2a-e illustrate FRY expression in breast cancer cell lines and a nontransformed human mammary epithelial cell line. (a) FRY mRNA expression levels and (b) FRY polypeptide expression levels in the non-tumorigenic MCF10A human mammary epithelial cell line and several ER-negative (HCC 1954, MDA-MB-231) and ER-positive (T47D, MCF7) human mammary epithelial cancer cell lines. (c,d) Fry expression levels in the human a breast cancer cell line with ectopic expression of wt Cop Fry (231wCFry) and without ectopic expression of Fry (MDA-MB-231): (c) Southern Blots portraying mRNA levels of wt Cop Fry (rat Fry) or human FRY in each cell line and graph depicting combined rat and human FRY mRNA expression, normalized to the expression of β-actin in each species; (d) Western Blot portraying total FRY polypeptide (rat and human) in the cell lines. (e) FRY polypeptide expression in the MCF10A cell lines stably transfected with either nontargeting shRNA (10A-CV) or FRY-targeting shRNA (10A-shFRY: 10A-1.1, 10A-1.2).

FIG. 3 illustrates a comparison of tumor sizes as a function of time after injection.

FIGS. 4a-k illustrate FRY expression in clinical breast cancer cohorts. (a) Analysis of microarray data available on the Oncomine 3.0 Cancer Profiling database (20) (Cancer=40; Normal=7) (p<0.0001); (b) Difference in pathologist ratings for nuclear FRY staining (Cancer=69; Normal=9) (p<0.02); (d) Nuclear staining of epithelial cells were scored by a pathologist. Each shaded bar represents the % of each tissue phenotype which was assigned a rating of four (indicating 70-100% of the nuclei in the sample stained positive for FRY); (g) Quantification of FRY polypeptide levels using quantitative image analysis software; (k) FRY polypeptide expression levels are lower in estrogen receptor negative (ER⁻) human breast tumors relative to estrogen receptor-positive (ER⁺) breast carcinomas (p<0.05).

FIG. 5 illustrates Venn diagrams of FRY-responsive genes.

FIGS. 6a-b depict the quantification of protein expression levels in isogenic cell line pairs: MDA-MB-231/231wCFry and 10A-CV/10A-shFRY. (a) Comparison of steady-state β-Catenin (an important protein in Wnt Signaling) expression levels in isogenic cell line pairs. (b) Comparison of steady-state α4-Integrin expression levels in isogenic cell line pairs.

FIG. 7 shows that FRY mRNA expression is decreased in prostate, ovarian, lung, brain and blood cancers.

FIGS. 8a-c illustrate that nuclear FRY protein expression was significantly higher in benign breast lesions compared to malignant lesions. (a) and (b) are floating bar charts designating min to max for each group with a line at the median. Pathologist scores are from 0-3 (0: <10% of epithelial cell nuclei were positive for FRY, 1: 10-40% of epithelial cell nuclei stained positive for FRY, 2: 40-70% of epithelial cell nuclei stained positive for FRY, 3: 70-100% of epithelial cell nuclei stained positive for FRY).

### DETAILED DESCRIPTION OF THE INVENTION

### 1. Overview

This invention generally relates to pharmaceutical compositions comprising FRY polypeptides and polynucleotides that are useful for the treatment of cancer. The invention further relates to the use of the pharmaceutical composition for the treatment of cancer, methods for determining the effectiveness of the treatment of cancer, methods for the diagnosis and prognosis of cancer and methods to differentiate stem cells.

Most breast cancers originate from normal epithelial cells and lose cell adhesion in the epithelial-to-mesenchymal transition process. In accordance with the present invention, in-vitro, in-vivo and in-silico analyses of isogenic mammary epithelial cell line pairs with altered FRY expression, confirmed that FRY is affecting epithelial cell differentiation, cell-cell adhesion, and cell mobility as well as numerous other pathways involved in epithelial cell development and function.

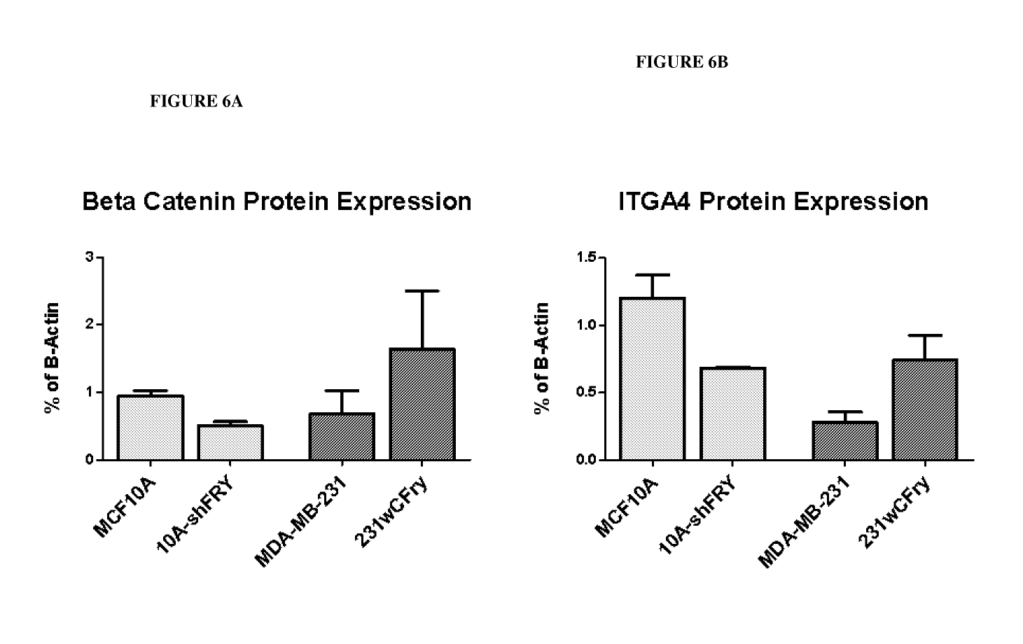

More than 85% of cancers arise from epithelial cells. The in silico, in vitro and in vivo observations support a role for FRY in epithelial cell differentiation. The mRNA expression profiles from more than eight hundred human cancers and normal tissues (not including breast cancers) available in the Oncomine 3.0 Cancer Profiling Database revealed that FRY is not only decreased in breast tumors relative to normal mammary tissues, but is also decreased in additional epithelial cell cancers including human prostate, ovarian, lung, and brain cancers relative to normal tissues as well as blood cancers (FIG. 6; Table S10). The results demonstrate that FRY is not only a mammary tumor suppressor gene and mammary cancer susceptibility gene, but is more generally an epithelial cell tumor suppressor and susceptibility gene. The expression of FRY enhances cell polarization, adhesion, and differentiation, and can reduce tumor growth and invasiveness.

### 2. Definitions

As used herein, the singular forms “a,” “an” and “the” include plural references unless the content clearly dictates otherwise.

The term “about”, as used here, refers to +/−10% of a value.

The term “FRY”, as used herein, means the mammalian homolog of the Drosophila melangoster furry gene, and refers to all isoforms and variants of a functional FRY polypeptide and the polynucleotide that encodes the functional FRY polypeptide.

The term “functional” refers to the structural properties of a polypeptide encoded by a gene, and includes a polypeptide that retains the necessary structural properties to perform the activity of the polypeptide encoded by the wild type gene. For example, a mutant FRY polypeptide with certain amino acid substitutions resulting from certain single nucleotide polymorphisms will not operate and function as a tumor suppressor polypeptide, and thus is not a functional FRY polypeptide.

The term “excipient” refers to any essentially accessory substance that may be present in the finished dosage form. For example, the term “excipient” includes vehicles, binders, disintegrants, fillers (diluents), suspending/dispersing agents, and so forth.

The term “antibody” refers to an immunoglobulin or antigen-binding fragment thereof, and encompasses any such polypeptide comprising an antigen-binding fragment of an antibody. The term includes but is not limited to polyclonal, monoclonal, monospecific, polyspecific, humanized, human, single-chain, single-domain, chimeric, synthetic, recombinant, hybrid, mutated, grafted, and in vitro generated antibodies. The term “antibody” also includes antigen-binding fragments of an antibody. Examples of antigen-binding fragments include, but are not limited to, Fab fragments (consisting of the V_{L}, V_{H}, C_{L} and C_{H}1 domains); Fd fragments (consisting of the V_{H} and C_{H}1 domains); Fv fragments (referring to a dimer of one heavy and one light chain variable domain in tight, non-covalent association); dAb fragments (consisting of a V_{H} domain); single domain fragments (V_{H} domain, V_{L} domain, V_{HH} domain, or V_{NAR} domain); isolated CDR regions; (Fab′)₂ fragments, bivalent fragments (comprising two Fab fragments linked by a disulphide bridge at the hinge region), scFv (referring to a fusion of the V_{L} and V_{H} domains, linked together with a short linker), and other antibody fragments that retain antigen-binding function.

As used herein, the term “epitope” refers to a site on an antigen to which B and/or T cells respond or a site on a molecule against which an antibody will be produced and/or to which an antibody will bind. For example, an epitope can be recognized by an antibody defining the epitope. An epitope can be either a “linear epitope” (where a primary amino acid primary sequence comprises the epitope; typically at least 3 contiguous amino acid residues, and more usually, at least 5, and up to about 8 to about 10 amino acids in a unique sequence) or a “conformational epitope” (an epitope wherein the primary, contiguous amino acid sequence is not the sole defining component of the epitope). A conformational epitope may comprise an increased number of amino acids relative to a linear epitope, as this conformational epitope recognizes a three-dimensional structure of the peptide or protein. For example, when a protein molecule folds to form a three dimensional structure, certain amino acids and/or the polypeptide backbone forming the conformational epitope become juxtaposed enabling the antibody to recognize the epitope. Methods of determining conformation of epitopes include but are not limited to, for example, x-ray crystallography, two-dimensional nuclear magnetic resonance spectroscopy and site-directed spin labeling and electron paramagnetic resonance spectroscopy. See, for example, Epitope Mapping Protocols in Methods in Molecular Biology, Vol. 66, Glenn E. Morris, Ed. (1996), the disclosure of which is incorporated in its entirety herein by reference.

The term “cancer” refers to or describes the physiological condition in mammals in which a population of cells is characterized by unregulated cell growth. Examples of cancer include, but are not limited to, epithelial cell cancer, breast cancer, prostate cancer, ovarian cancer, lung cancer, brain cancer, blood cancer, carcinoma, lymphoma, blastoma, sarcoma, and leukemia. More particular examples of such cancers include squamous cell cancer, small-cell lung cancer, non-small cell lung cancer, adenocarcinoma of the lung, squamous carcinoma of the lung, cancer of the peritoneum, hepatocellular cancer, gastrointestinal cancer, pancreatic cancer, glioblastoma, cervical cancer, liver cancer, bladder cancer, hepatoma, colon cancer, colorectal cancer, endometrial or uterine carcinoma, salivary gland carcinoma, kidney cancer, liver cancer, prostate cancer, vulval cancer, thyroid cancer, hepatic carcinoma and various types of head and neck cancers.

The term “tumor” as used herein refers to any mass of tissue that results from excessive cell growth or proliferation, either benign (noncancerous) or malignant (cancerous), including pre-cancerous lesions.

The term “metastasis” as used herein refers to the process by which a cancer spreads or transfers from the site of origin to other regions of the body with the development of a similar cancerous lesion at the new location. A “metastatic” or “metastasizing” cell is one that loses adhesive contacts with neighboring cells and migrates via the bloodstream or lymph from the primary site of disease to invade neighboring body structures.

The terms “cancer cell,” and “tumor cell,” and grammatical equivalents refer to the total population of cells derived from a tumor or a pre-cancerous lesion.

As used herein, the terms “biopsy” and “biopsy tissue” refer to a sample of tissue or fluid that is removed from a subject for the purpose of determining if the sample contains cancerous tissue. In some embodiments, biopsy tissue or fluid is obtained because a subject is suspected of having cancer, and the biopsy tissue or fluid is then examined for the presence or absence of cancer.

As used herein, the term “subject” refers to any animal (e.g., a mammal), including, but not limited to humans, non-human primates, rodents, and the like, which is to be the recipient of a particular treatment. Typically, the terms “subject” and “patient” are used interchangeably herein in reference to a human subject.

The term “effective amount,” “therapeutically effective amount” or “therapeutic effect” refers to an amount of an antibody, polypeptide, polynucleotide, small organic molecule, or other drug effective to “treat” a disease or disorder in a subject or mammal. In the case of cancer, the therapeutically effective amount of the drug has a therapeutic effect and as such can reduce the number of cancer cells; decrease tumorigenicity, tumorigenic frequency or tumorigenic capacity; reduce the number or frequency of cancer cells; reduce the tumor size; inhibit or stop cancer cell infiltration into peripheral organs including, for example, the spread of cancer into soft tissue and bone; inhibit and stop tumor metastasis; inhibit and stop tumor growth; relieve to some extent one or more of the symptoms associated with the cancer; reduce morbidity and mortality; improve quality of life; or a combination of such effects.

Terms such as “treating” or “treatment” or “to treat” or “alleviating” or “to alleviate” refer to both 1) therapeutic measures that cure, slow down, lessen symptoms of, and/or halt progression of a diagnosed pathologic condition or disorder and 2) prophylactic or preventative measures that prevent and/or slow the development of a targeted pathologic condition or disorder. Thus those in need of treatment include those already with the disorder; those prone to have the disorder; and those in whom the disorder is to be prevented. In certain embodiments, a subject is successfully “treated” according to the methods of the present invention if the patient shows one or more of the following: a reduction in the number of or complete absence of cancer cells; a reduction in the tumor size; inhibition of or an absence of cancer cell infiltration into peripheral organs including, for example, the spread of cancer into soft tissue and bone; inhibition of or an absence of tumor metastasis; inhibition or an absence of tumor growth; relief of one or more symptoms associated with the specific cancer; reduced morbidity and mortality; improvement in quality of life; reduction in tumorigenicity; reduction in the number or frequency of cancer cells; or some combination of effects.

As used herein, the terms “polynucleotide” or “nucleic acid” refer to a polymer composed of a multiplicity of nucleotide units (ribonucleotide or deoxyribonucleotide or related structural variants) linked via phosphodiester bonds, including but not limited to, DNA or RNA. The term encompasses sequences that include any of the known base analogs of DNA and RNA. Examples of a polynucleotide include and are not limited to mRNA, miRNA, tRNA, rRNA, snRNA, siRNA, dsRNA, cDNA and DNA/RNA hybrids.

The term “gene” refers to a nucleic acid (e.g., DNA) sequence that comprises coding sequences necessary for the production of a polypeptide, precursor, or RNA (e.g., rRNA, tRNA). The polypeptide can be encoded by a full length coding sequence or by any portion of the coding sequence so long as the desired activity or functional properties (e.g., enzymatic activity, ligand binding, signal transduction, immunogenicity, etc.) of the full-length polypeptide or fragment are retained. The term also encompasses the coding region of a structural gene and the sequences located adjacent to the coding region on both the 5′ and 3′ ends for a distance of about 1 kb or more on either end such that the gene corresponds to the length of the full-length mRNA. The term “gene” encompasses both cDNA and genomic forms of a gene.

The terms “polypeptide”, “peptide”, “protein”, and “protein fragment” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymers.

The term “amino acid” refers to naturally occurring and synthetic amino acids, as well as amino acid analogs and amino acid mimetics that function similarly to the naturally occurring amino acids. Naturally occurring amino acids are those encoded by the genetic code, as well as those amino acids that are later modified, e.g., hydroxyproline, gamma-carboxyglutamate, and O-phosphoserine. “Amino acid analogs” refers to compounds that have the same basic chemical structure as a naturally occurring amino acid, e.g., an alpha carbon that is bound to a hydrogen, a carboxyl group, an amino group, and an R group, e.g., homoserine, norleucine, methionine sulfoxide, methionine methyl sulfonium. Such analogs can have modified R groups (e.g., norleucine) or modified peptide backbones, but retain the same basic chemical structure as a naturally occurring amino acid. “Amino acid mimetics” refers to chemical compounds that have a structure that is different from the general chemical structure of an amino acid, but that functions similarly to a naturally occurring amino acid.

“Conservatively modified variants” applies to both amino acid and nucleic acid sequences. “Amino acid variants” refers to amino acid sequences. With respect to particular nucleic acid sequences, conservatively modified variants refers to those nucleic acids which encode identical or essentially identical amino acid sequences, or where the nucleic acid does not encode an amino acid sequence, to essentially identical or associated (e.g., naturally contiguous) sequences. Because of the degeneracy of the genetic code, a large number of functionally identical nucleic acids encode most proteins. For instance, the codons GCA, GCC, GCG and GCU all encode the amino acid alanine. Thus, at every position where an alanine is specified by a codon, the codon can be altered to another of the corresponding codons described without altering the encoded polypeptide. Such nucleic acid variations are “silent variations”, which are one species of conservatively modified variations. Every nucleic acid sequence herein which encodes a polypeptide also describes silent variations of the nucleic acid. One of skill will recognize that in certain contexts each codon in a nucleic acid (except AUG, which is ordinarily the only codon for methionine, and TGG, which is ordinarily the only codon for tryptophan) can be modified to yield a functionally identical molecule. Accordingly, silent variations of a nucleic acid which encodes a polypeptide is implicit in a described sequence with respect to the expression product.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant”, including where the alteration results in the substitution of an amino acid with a chemically similar amino acid. Conservative substitution tables providing functionally similar amino acids are well known in the art (See, for example, Table 1). Guidance concerning which amino acid changes are likely to be phenotypically silent can also be found in Bowie et al., 1990, Science 247:1306 1310. Such conservatively modified variants are in addition to and do not exclude polymorphic variants, interspecies homologs, and alleles. Typical conservative substitutions include but are not limited to: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)). Amino acids can be substituted based upon properties associated with side chains, for example, amino acids with polar side chains may be substituted, for example, Serine (S) and Threonine (T); amino acids based on the electrical charge of a side chains, for example, Arginine (R) and Histidine (H); and amino acids that have hydrophobic side chains, for example, Valine (V) and Leucine (L). As indicated, changes are typically of a minor nature, such as conservative amino acid substitutions that do not significantly affect the folding or activity of the protein.

A “recombinant” peptide, polypeptide, or protein refers to a peptide, polypeptide, or protein produced by recombinant DNA techniques; i.e., produced from cells transformed by an exogenous DNA construct encoding the desired peptide, polypeptide, or protein.

A “fusion polypeptide” refers to a polypeptide created through the joining of two or more heterologous proteins or polypeptides. A heterologous protein, polypeptide, nucleic acid, or gene is one that originates from a foreign species, or, if from the same species, is substantially modified from its original form. Two fused domains or sequences are heterologous to each other if they are not adjacent to each other in a naturally occurring protein or nucleic acid.

The term “predetermined standard” refers to a control level of a particular protein expressed in samples of the same type of tissue or cells from subjects who do not have cancer; for example, a predetermined standard can be a control level determined based upon the expression of the FRY gene in breast tissue isolated from subjects who do not have breast cancer.

The term “phenotype” as used herein, refers to an observable characteristic or trait of an organism (e.g. stem cell) such as its morphology, development, biochemical or physiological properties, or behavior. Phenotypes result from the expression of an organism's genes as well as the influence of environmental factors and the interactions between the two. Examples of a stem cell phenotype include, but are in no way limited to, size, cell surface marker profile, proliferation potential, immunogenicity, uncontrolled growth (i.e. tumor cells), plasticity (i.e. differentiation potential), engraftment potential, therapeutic potential, and combinations thereof.

The term “hormone receptor negative cancer” as used herein refers to a type of cancer wherein the cancer cell does not express a receptor for a type of hormone, and typically does not require the hormone to grow. An example of a hormone receptor negative cancer is estrogen receptor negative, which describes cells that do not have a receptor to which the hormone estrogen will bind. Cancer cells that are estrogen receptor negative do not need estrogen to grow, and usually do not stop growing when treated with hormones that block estrogen from binding. Another example is a triple negative breast cancer, which refers to breast cancer that does not express the genes for estrogen receptor (ER), progesterone receptor (PR) or human epidermal growth factor receptor 2 (Her2).

The term “probe” as used herein refers to an oligonucleotide capable of binding to a target nucleic acid of complementary sequence through one or more types of chemical bonds, usually through complementary base pairing, usually through hydrogen bond formation. Probes may bind target sequences lacking complete complementarity with the probe sequence depending upon the stringency of the hybridization conditions. There may be any number of base pair mismatches which will interfere with hybridization between the target sequence and the single stranded nucleic acids described herein. However, if the number of mutations is so great that no hybridization can occur under even the least stringent of hybridization conditions, the sequence is not a complementary target sequence. A probe may be single stranded or partially single and partially double stranded. The strandedness of the probe is dictated by the structure, composition, and properties of the target sequence. Probes may be directly labeled or indirectly labeled such as with biotin to which a streptavidin complex may later bind.

“Complement” or “complementary” as used herein to refer to a nucleic acid may mean Watson-Crick (e.g., A-T/U and C-G) or Hoogsteen base pairing between nucleotides or nucleotide analogs of nucleic acid molecules. A full complement or fully complementary may mean 100% complementary base pairing between nucleotides or nucleotide analogs of nucleic acid molecules.

“Stringent hybridization conditions” as used herein refers to conditions under which a first nucleic acid sequence (e.g., probe) hybridizes to a second nucleic acid sequence (e.g., target), such as in a complex mixture of nucleic acids. Stringent conditions are sequence-dependent and may vary in different circumstances, and can be suitably selected by one skilled in the art. Stringent conditions may be selected to be about 5-10° C. lower than the thermal melting point (Tm) for the specific sequence at a defined ionic strength pH. The Tm may be the temperature (under defined ionic strength, pH, and nucleic concentration) at which 50% of the probes complementary to the target hybridize to the target sequence at equilibrium (as the target sequences are present in excess, at Tm, 50% of the probes are occupied at equilibrium). Stringent conditions may be those in which the salt concentration is less than about 1.0 M sodium ion, such as about 0.01-1.0 M sodium ion concentration (or other salts) at pH 7.0 to 8.3 and the temperature is at least about 30° C. for short probes (e.g., about 10-50 nucleotides) and at least about 60° C. for long probes (e.g., greater than about 50 nucleotides). Stringent conditions may also be achieved with the addition of destabilizing agents such as formamide. For selective or specific hybridization, a positive signal may be at least 2 to 10 times background hybridization. Exemplary stringent hybridization conditions include the following: 50% formamide, 5×SSC, and 1% SDS, incubating at 42° C., or, 5×SSC, 1% SDS, incubating at 65° C., with wash in 0.2×SSC, and 0.1% SDS at 65° C. However, several factors other than temperature, such as salt concentration, can influence the stringency of hybridization and one skilled in the art can suitably select the factors to accomplish a similar stringency.

“Substantially identical” as used herein refers to that the nucleic or amino acid sequence is at least 60%, 65%, 70%, 75%, 80%, 85%, 90%, 95%, 97%, 98% or 99% identical over a region of 8, 9, 10, 11, 12, 13, 14, 15, 16, 17, 18, 19, 20, 21, 22, 23, 24, 25, 30, 35, 40, 45, 50 or more nucleotides or amino acids, or with respect to nucleic acids, if the first sequence is substantially complementary to the complement of the second sequence. Preferably, such variant nucleic acid and polypeptide sequences will share 75% or more (i.e. 80, 85, 90, 95, 97, 98, 99% or more) sequence identity with the sequences recited in the application. Preferably such sequence identity is calculated with regard to the full length of the reference sequence (i.e. the sequence recited in the application).

“Substantially complementary” as used herein refers to that the nucleic acid sequence is at least 60%, 65%, 70%, 75%, 80%, 85%, 90%, 95%, 97%, 98% or 99% identical to the complement of a second sequence over a region of 8, 9, 10, 11, 12, 13, 14, 15, 16, 17, 18, 19, 20, 21, 22, 23, 24, 25, 30, 35, 40, 45, 50 or more nucleotides, or that the two sequences hybridize under stringent hybridization conditions.

As used herein, “targeting agents” refer to ligands, polymers, proteins, cytokines, chemokines, peptides, nucleic acids, lipids, saccharides or polysaccharides, small molecules or any combination thereof, (for example a gylcolipid, glycoprotein etc) that bind to a receptor or other molecule on the surface of a targeted cell. An exemplary small-molecule targeting compound is folate, which targets the folate receptor. The degree of specificity can be modulated through the selection of the targeting molecule. For example, antibodies are very specific. These can be polyclonal, monoclonal, fragments, recombinant, or single chain, many of which are commercially available or readily obtained using standard techniques. Examples of antibodies include, but not limited to abciximab, basiliximab, cetuximab, infliximab, rituximab, trastuzumab etc.

The term “transfection” refers to the uptake of foreign DNA by a cell. A cell has been “transfected” when exogenous (i.e., foreign) DNA has been introduced inside the cell membrane. Transfection can be either transient (i.e., the introduced DNA remains extrachromosomal and is diluted out during cell division) or stable (i.e., the introduced DNA integrates into the cell genome or is maintained as a stable episomal element).

“Cotransfection” refers to the simultaneous or sequential transfection of two or more vectors into a given cell.

The term “promoter element” or “promoter” or “regulatory region” refers to a DNA regulatory region capable of being bound by an RNA polymerase in a cell (e.g., directly or through other promoter-bound proteins or substances) and allowing for the initiation of transcription of a coding or non-coding RNA sequence. A promoter sequence is, in general, bounded at its 3′ terminus by the transcription initiation site and extends upstream (5′ direction) to include the minimum number of bases or elements necessary to initiate transcription at any level. Within the promoter sequence may be found a transcription initiation site (conveniently defined, for example, by mapping with nuclease S1), as well as protein binding domains (consensus sequences) responsible for the binding of RNA polymerase. The promoter may be operably associated with other expression control sequences, including enhancer and repressor sequences.

The term “in operable combination”, “in operable order” or “operably linked” refers to the linkage of nucleic acid sequences in such a manner that a nucleic acid molecule capable of directing the transcription of a given gene and/or the synthesis of a desired protein molecule is produced. The term also refers to the linkage of amino acid sequences in such a manner so that a functional protein is produced.

A “test sample” or a “biological sample” as used herein may mean a sample of biological tissue or fluid that comprises nucleic acids and/or polypeptides. Such samples include, but are not limited to, tissue isolated from animals. Biological samples may also include sections of tissues such as biopsy and autopsy samples, frozen sections taken for histological purposes, blood, plasma, serum, sputum, stool, tears, mucus, urine, effusions, amniotic fluid, ascitic fluid, hair, and skin. Biological samples also include explants and primary and/or transformed cell cultures derived from patient tissues. A biological sample may be provided by removing a sample of cells from an animal, but can also be accomplished by using previously isolated cells (e.g., isolated by another person, at another time, and/or for another purpose), or by performing the methods described herein in vivo. Archival tissues, such as those having treatment or outcome history, may also be used.

The term “vector” refers to a nucleic acid assembly capable of transferring gene sequences to target cells (e.g., viral vectors, non-viral vectors, particulate carriers, and liposomes). The term “expression vector” refers to a nucleic acid assembly containing a promoter which is capable of directing the expression of a sequence or gene of interest in a cell. Vectors typically contain nucleic acid sequences encoding selectable markers for selection of cells that have been transfected by the vector. Generally, “vector construct,” “expression vector,” and “gene transfer vector,” refer to any nucleic acid construct capable of directing the expression of a gene of interest and which can transfer gene sequences to target cells. Thus, the term includes cloning and expression vehicles, as well as viral vectors.

### 3. Pharmaceutical Compositions and Methods of Use

The present invention provides pharmaceutical compositions comprising at least one FRY polypeptide or at least one FRY polynucleotide encoding a FRY polypeptide and a pharmaceutically acceptable carrier. To administer the pharmaceutical composition to a subject, it is preferable to formulate the molecules in a composition comprising one or more pharmaceutically acceptable carriers. The phrase “pharmaceutically acceptable” refers to molecular entities and compositions that do not produce allergic, or other adverse reactions when administered using routes well-known in the art. “Pharmaceutically acceptable carriers” include any and all clinically useful solvents, dispersion media, coatings, antibacterial and antifungal agents, isotonic and absorption delaying agents and the like.

The pharmaceutical composition may comprise a FRY polypeptide that is substantially identical to SEQ ID NO. 2. The FRY polypeptide may also be a conservatively modified variant of SEQ ID NO. 2. One skilled in the art can identify the amino acid conservative substitutions as well as determine the length of the polypeptide to maintain the activity of the FRY polypeptide utilizing routine methods known in the art.

The FRY polypeptide can be an isolated or purified protein. The FRY polypeptide may also be a fusion protein. The FRY polypeptide can be fused to targeting agents such as an antibody, an antibody fragment or a peptide ligand targeted to a specific receptor. Examples include, but not limited to linear peptides with RGD motif, peptides with EGF motif, a cell penetrating peptide such as TAT. For additional guidance, skilled artisans may consult Ausubel et al. (supra), Sambrook et al. (Molecular Cloning, A Laboratory Manual, Cold Spring Harbor Press, Cold Spring Harbor, N.Y., 1989). Alternatively, the peptides/polypeptides/proteins of the invention can be chemically synthesized (see e.g., Creighton, Proteins: Structures and Molecular Principles, W.H. Freeman & Co., NY, 1983).

An “isolated” or “purified” peptide, polypeptide, or protein refers to a peptide, polypeptide, or protein that has been separated from other proteins, lipids, and nucleic acids with which it is naturally associated. The polypeptide/protein can constitute at least 10% (i.e., any percentage between 10% and 100%, e.g., 20%, 30%, 40%, 50%, 60%, 70%, 80%, 85%, 90%, 95%, and 99%) by dry weight of the purified preparation. Purity can be measured by any appropriate standard method, for example, by column chromatography, polyacrylamide gel electrophoresis, or HPLC analysis. An isolated polypeptide/protein described in the invention can be purified from a natural source, produced by recombinant DNA techniques, or by chemical methods.

The FRY polypeptide may also be conjugated to a targeting agent. Covalent attachment is achieved by incorporating mutually reactive functional groups on the therapeutic agent and FRY polypeptide or by using homo- and/or hetero-multifunctional cross linkers. The examples of degradable covalent bonds include, but not limited to, enzyme-sensitive peptide linkers and bonds; auto-degradable ester and thioester bonds; acid-sensitive bonds like imines, hydrazones, carboxylic hydrazones, ketal, acetal, cis-aconityl, and trityl bonds; hypoxia-sensitive linkers; and self-immolative bonds. BIOCONJUGATE TECHNIQUES (Academic Press; 1st edition, Greg T. Hermanson, 1996) describes techniques for modifying or cros slinking of biomolecules. BIOCONJUGATE TECHNIQUES (Academic Press; 1st edition, Greg T. Hermanson, 1996) describes techniques for modifying or cros slinking of biomolecules.

The FRY polypeptide may also be PEGylated to improve the half life and stability of the polypeptide. The FRY polypeptide may also be conjugated to biodegradable polymers to improve the stability of the polypeptide, examples include polylactic and/or polyglycolic acids, polyanhydrides, polycaprolactones, polyethylene oxides, polybutylene terephthalates, starches, cellulose, chitosan, and/or any combinations of these.

The pharmaceutical composition may comprise a polynucleotide encoding a FRY polypeptide. The FRY polynucleotide may be substantially identical to SEQ ID NO. 1. The FRY polynucleotide may be substantially complementary to SEQ ID NO. 1. The FRY polynucleotide may also be a conservatively modified variant of SEQ ID NO. 1. One skilled in the art can select as well as identify nucleic acid conservative substitutions as well as determine the length of the polynucleotide to maintain the activity of the FRY polypeptide utilizing routine methods known in the art.

A person of ordinary skill in the art would recognize that a polynucleotide sequence encoding a FRY polypeptide may be incorporated into different vectors for delivery to cancer cells or delivered as a naked (vectorless) DNA. The pharmaceutical composition comprising the FRY polynucleotide may be delivered by using a delivery device such as a catheter. Other delivery devices are known to those with skill in the art.

The vectors suitable for hosting the FRY polynucleotides of the present invention include, without limitations, plasmid vectors and viral vectors. See Daya, S et. al, “Gene Therapy Using Adeno-Associated Virus Vectors”, Clinical Microbiology Reviews October 2008, p. 583-593 Vol. 21, No. 4. The methods of the present invention utilize routine techniques in the field of molecular biology. Basic texts disclosing general molecular biology methods include Sambrook et al., Molecular Cloning, A Laboratory Manual (3d ed. 2001) and Ausubel et al., Current Protocols in Molecular Biology (1994).

In one embodiment, the vector comprises an adeno-associated virus (AAV), from the parvovirus family. A person of ordinary skill in the art will recognize that among the advantages of AAV are the facts that AAV is not pathogenic and that most people treated with AAV will not build an immune response to remove the virus.

Both adenoviral and AAV vectors have been shown to be effective at delivering transgenes (including transgenes directed to desired target genes) into central nervous system cells. See, e.g., Bankiewicz et al., “Long-Term Clinical Improvement in MPTP-Lesioned Primates after Gene Therapy with AAV-hAADC”, Mol. Ther., E-publication Jul. 6, 2006 (A combination of intrastriatal AAV containing a nucleic sequence encoding L-amino acid decarboxylase inhibitor (AAV-hAADC) gene therapy and administration of the dopamine precursor 1-Dopa to MPTP-lesioned monkeys, resulted in long-term improvement in clinical rating scores, significantly lowered 1-Dopa requirements, and a reduction in 1-Dopa-induced side effects); Machida et al., Biochem Biophys Res Commun. 343(1):190-7 (2006) (Reporting a direct inhibition of mutant gene expression by rAAV-mediated delivery of RNAi into the HD model mouse striatum after the onset of disease); Mittoux et al., J. Neurosci. 22(11):4478-86 (2002). (Adenovirus-mediated ciliary neurotrophic factor delivery to brain resulted in increased survival of striatal neurons in response to a neurotoxin).

Examples of other vectors for the delivery of the FRY nucleotide to a cancer cell include and not limited to, a lentivirus vector, a pox virus vector, and alphavirus vector, and a herpes virus vector. The vectors may be further designed to be expressed in certain types of cells according to the regulatory region chosen for the vector. The regulatory sequences may be organ and/or tissue specific promoters. By way of example, Mellon et al. was able to develop clonal, differentiated, neurosecretory cell line comprising hypothalamic neurons secreting gonadotropin release factor by creating transgenic mice comprising SV40 T-antigen oncogene under control of GnRH regulatory region. Neuron, 5(1):1-10 (1990). For additional guidance, skilled artisans may consult Ausubel et al. (supra), Sambrook et al. (Molecular Cloning, A Laboratory Manual, Cold Spring Harbor Press, Cold Spring Harbor, N.Y., 1989). Other methods for producing retroviruses and for infecting cells in vitro or in vivo are described in Current Protocols in Molecular Biology, Ausubel, F. M. et al. (eds.) Greene Publishing Associates, (1989), Sections 9.10-9.14.

The pharmaceutical composition may further comprise additional anti-neoplastic agents to treat cancer or be co-administered with other anti-neoplastic agents. Examples include but not limited to, Abarelix, Aldesleukin, Alemtuzumab, Alitretinoin, Allopurinol, Altretamine, Amifostine, Anastrozole, arsenic trioxide, asparaginase, azacitidine, bevacuzimab, bleomycin, bortezomib, capecitabine, carboplatin, carmustine, celecoxib, cetuximab, chlorambucil, cisplatin, cladribine, clofarabine, cyclophosphamide, cytarabine, dacarbazine, daunorubicin, daunomycin, dexrazoxane, docetaxel, doxorubicin, epirubicin, Epoetin alfa, Erlotinib, Estramustine, etoposide phosphate, etoposide, VP-16, exemestane, Filgrastim, Floxuridine, Fludarabine, fluorouracil, 5-FU, fulvestrant, gefitinib, gemcitabine, gemtuzumab, goserelin acetate, histrelin acetate, hydroxyurea, idarubicin, ifosfamide, imatinib mesylate, interferon alfa 2a, irinotecan, lenalidomide, letrozole, leucovorin, Leuprolide Acetate, Levamisole, lomustine, CCNU, meclorethamine, nitrogen mustard, megestrol acetate, melphalan, L-PAM, mercaptopurine, 6-MP, mesna, methotrexate, methoxsalen, mitomycin C, mitotane, mitoxantrone, nelarabine, Nofetumomab, Oprelvekin, Oxaliplatin, Paclitaxel, Palifermin, Pamidronate, Pegademase, pemetrexed disodium, pentostatin, pipobroman, plicamycin, mithramycin, porfimer sodium, procarbazine, quinacrine, Rasburicase, Rituximab, Sargramostim, Sorafenib, Streptozocin, sunitinib maleate, tamoxifen, temozolomide, teniposide, VM-26, testolactone, thioguanine, 6-TG, thiotepa, topotecan, toremifene, Tositumomab, Trastuzumab, Uracil Mustard, Valrubicin, Vinblastine, Vincristine, Vinorelbine, Zoledronate, and zoledronic acid.

Examples of pharmaceutically acceptable carriers or additives include water, a pharmaceutical acceptable organic solvent, collagen, polyvinyl alcohol, polyvinylpyrrolidone, a carboxyvinyl polymer, carboxymethylcellulose sodium, polyacrylic sodium, sodium alginate, water-soluble dextran, carboxymethyl starch sodium, pectin, methyl cellulose, ethyl cellulose, xanthan gum, gum Arabic, casein, gelatin, agar, diglycerin, glycerin, propylene glycol, polyethylene glycol, Vaseline, paraffin, stearyl alcohol, stearic acid, human serum albumin (HSA), mannitol, sorbitol, lactose, a pharmaceutically acceptable surfactant and the like. Additives used are chosen from, but not limited to, the above or combinations thereof, as appropriate, depending on the dosage form of the present invention.

When the pharmaceutical composition of the present invention is used as a medicament, the compound of the present invention is mixed with a pharmaceutically acceptable carrier (excipient, binder, disintegrant, corrigent, flavor, emulsifier, diluent, solubilizing agents and the like) to give a pharmaceutical composition which can be orally or parenterally administered. A pharmaceutical composition can be formulated by a general method.

The invention provides a method for treating cancer administering to a subject in need thereof an effective amount of a pharmaceutical composition comprising a FRY polypeptide or a FRY polynucleotide encoding a FRY polypeptide. The invention also provides pharmaceutical compositions for the suppression of cancer metastasis in a subject in need thereof.

The type of cancer is characterized by the under expression of FRY and/or the lack of expression of a functional FRY polypeptide. In general, the lack of expression of the functional FRY polypeptide is defined as a retention of less than 50% of FRY function in a non-cancerous cell of the same type. To generate a predetermined standard, straightforward tests for determination of the level of FRY polypeptide present, expressed, and/or activity of normal cells are known in the art (e.g., western blotting, immunohistochemisty and sequencing).

Generally the cancer can be characterized as cancers that arise from epithelial cells. The cancer cells may also possess a stem cell phenotype. In one embodiment, the cancer is breast cancer, a hormone receptor negative breast cancer or a triple negative breast cancer, in which the breast cancer cells lack the estrogen receptor (ER), the progersterone receptor (PR) and the human epidermal growth factor receptor 2 (Her2).

Other types of cancers characterized by these conditions may be selected from among solid tumors or liquid cancers. Solid tumors include, without limitations, prostate cancer, ovarian, lung, brain cancer, squamous cell cancer, small-cell lung cancer, non-small cell lung cancer, adenocarcinoma of the lung, squamous carcinoma of the lung and the like. In other embodiments, different cancers of blood cells are amenable to treatment. These blood cancers include, without limitations, Acute lymphocytic leukemia, Chronic myelogenous leukemia, Chronic lymphocytic leukemia, Hairy cell leukemia, myelomas and lymphomas. In other embodiments, other types of cancer include carcinomas, such as adenocarcinoma, squamous cell carcinoma, adenosquamous carcinoma, melanoma, anaplastic carcinoma, anaplastic or undifferentiated carcinomas.

Depending on the nature of the cancer, the pharmaceutical compositions of the instant invention may be administered by routes independently selected from the group consisting of oral administration, intravenous administration, intraarterial administration, intramuscular administration, intracolonic administration, intracranial administration, intrathecal administration, intraventricular administration, intraurethral administration, intravaginal administration, subcutaneous administration, intraocular administration, intranasal administration, and any combinations thereof.

In the present specification, parenteral includes subcutaneous injection, intravenous injection, intramuscular injection, intraperitoneal injection, drip or topical administration (transdermal administration, transocular administration, transpulmonary or bronchial administration, transnasal administration, transrectal administration and the like) and the like.

The dose of the pharmaceutical composition of the present invention is determined according to the age, body weight, general health condition, sex, diet, administration time, administration method, clearance rate, and the level of disease for which patients are undergoing treatments at that time, or further in consideration of other factors. While the daily dose of the compound of the present invention varies depending on the condition and body weight of patient, the kind of the compound, administration route and the like, it is parenterally administered at, for example, 0.01 to 100 mg/patient/day by subcutaneous, intravenous, intramuscular, transdermal, transocular, transpulmonary or bronchial, transnasal or rectal administration.

Oral dosage forms may include capsules, tablets, emulsions and aqueous suspensions, dispersions, and solutions. In the case of tablets, commonly used carriers include, but are not limited to, lactose and corn starch. Lubricating agents, such as, but not limited to, magnesium stearate, also are typically added. For oral administration in a capsule form, useful diluents include, but are not limited to, lactose and dried corn starch. When aqueous suspensions or emulsions are administered orally, the active ingredient can be suspended or dissolved in an oily phase combined with emulsifying or suspending agents. If desired, certain sweetening, flavoring, or coloring agents can be added.

In particular examples, an oral dosage range is from about 1.0 to about 100 mg/kg body weight administered orally in single or divided doses, including from about 1.0 to about 50 mg/kg body weight, from about 1.0 to about 25 mg/kg body weight, from about 1.0 to about 10 mg/kg body weight (assuming an average body weight of approximately 70 kg; values adjusted accordingly for persons weighing more or less than average). For oral administration, the compositions are, for example, provided in the form of a tablet containing from about 50 to about 1000 mg of the active ingredient, particularly about 75 mg, about 100 mg, about 200 mg, about 400 mg, about 500 mg, about 600 mg, about 750 mg, or about 1000 mg of the active ingredient for the symptomatic adjustment of the dosage to the subject being treated.

### 5. Methods for the Diagnosis, the Prognosis of Cancer and Effectiveness of Treatment

This invention also provides diagnostic methods and methods to assess the prognosis of a cancer. A subject having cancer or prone to it can be determined based on the expression levels, patterns, or profile of the FRY gene, such as nucleic acids (e.g., mRNA, miRNA) or polypeptides in a test sample from the subject compared to a predetermined standard or standard level in a corresponding non-cancerous sample. In other words, FRY polypeptides and nucleic acids can be used as markers to indicate the presence or absence of cancer or the risk of having cancer, as well as to assess the prognosis of the cancer. Diagnostic and prognostic assays of the invention include methods for assessing the expression level of the nucleic acids or polypeptides. The methods and kits allow one to detect the type of cancer and stage of cancer. For example, a relative increase in the gene expression level of FRY may be indicative of the decrease in the size of a tumor.

The Bloom-Richardson-Elston grading system provides a breast cancer prognosis classification system to grade breast cancers. Lower grade tumors are associated with a good prognosis, and can be treated less aggressively, and a subject has a better survival rate. Higher grade tumors are associated with a bad prognosis, and are treated more aggressively. A grade or score is assigned to the cancer based upon many variables, such as the aggressiveness of the cancer, the spread of the cancer, other variables are considered by those with ordinary skill in the art. One with ordinary skill in the art can develop a cancer grading system based on the expression level of FRY by comparing various tumors according to the appropriate cancer grading system and the expression of FRY in each grade of a tumor. For example, the expression level of FRY can be determined in a Grade 1 breast cancer cell, in a Grade 2 breast cancer cell, etc. Thus when a breast tumor sample is obtained from a subject, the expression level of FRY can determine the grade of the breast cancer and a prognosis for the cancer can be provided to the subject. Examples of cancer grading systems include the TNM Classification of Malignant Tumours and the Gleason Grading system for prostate cancer.

The presence, level, or absence of the nucleic acid or polypeptide in a test sample can be evaluated by obtaining a test sample from a test subject and contacting the test sample with a compound or an agent capable of detecting the polypeptide or nucleic acid (e.g., mRNA, miRNA or genomic DNA probe). The “test sample” includes tissues, cells and biological fluids isolated from a subject, as well as tissues, cells and fluids present within a subject. The level of expression of a gene(s) of interest can be measured in a number of ways, including measuring the mRNA encoded by the gene; measuring the amount of polypeptide encoded by the gene; or measuring the activity of polypeptide encoded by the gene.

Expressed RNA samples can be isolated from biological samples using any of a number of well-known procedures. For example, biological samples can be lysed in a guanidinium-based lysis buffer, optionally containing additional components to stabilize the RNA. In some embodiments, the lysis buffer can contain purified RNAs as controls to monitor recovery and stability of RNA from cell cultures. Examples of such purified RNA templates include the Kanamycin Positive Control RNA from PROMEGA (Madison, Wis.), and 7.5 kb Poly(A)-Tailed RNA from LIFE TECHNOLOGIES (Rockville, Md.). Lysates may be used immediately or stored frozen at, e.g., −80° C.

Optionally, total RNA can be purified from cell lysates (or other types of samples) using silica-based isolation in an automation-compatible, 96-well format, such as the RNEASY purification platform (QIAGEN, Inc., Valencia, Calif.). Alternatively, RNA is isolated using solid-phase oligo-dT capture using oligo-dT bound to microbeads or cellulose columns. This method has the added advantage of isolating mRNA from genomic DNA and total RNA, and allowing transfer of the mRNA-capture medium directly into the reverse transcriptase reaction. Other RNA isolation methods are contemplated, such as extraction with silica-coated beads or guanidinium. Further methods for RNA isolation and preparation can be devised by one skilled in the art.

The methods of the present invention can also be performed using crude cell lysates, eliminating the need to isolate RNA. RNAse inhibitors are optionally added to the crude samples. When using crude cellular lysates, it should be noted that genomic DNA can contribute one or more copies of a target sequence, e.g., a gene, depending on the sample. In situations in which the target sequence is derived from one or more highly expressed genes, the signal arising from genomic DNA may not be significant. But for genes expressed at low levels, the background can be eliminated by treating the samples with DNAse, or by using primers that target splice junctions for subsequent priming of cDNA or amplification products. For example, one of the two target-specific primers could be designed to span a splice junction, thus excluding DNA as a template. As another example, the two target-specific primers can be designed to flank a splice junction, generating larger PCR products for DNA or unspliced mRNA templates as compared to processed mRNA templates. One skilled in the art could design a variety of specialized priming applications that would facilitate use of crude extracts as samples for the purposes of this invention.

The level of mRNA corresponding to a gene in a cell can be determined both in situ and in vitro. Messenger RNA isolated from a test sample can be used in hybridization or amplification assays that include, Southern or Northern analyses, PCR analyses, and probe arrays. A preferred diagnostic method for the detection of mRNA levels involves contacting the isolated mRNA with a nucleic acid probe that can hybridize to the mRNA encoded by the gene. The probe can be a full-length nucleic acid or a portion thereof, such as an oligonucleotide of at least 10 nucleotides in length and sufficient to specifically hybridize under stringent conditions to the mRNA.

In one format, mRNA (or cDNA prepared from it) is immobilized on a surface and contacted with the probes, for example, by running the isolated mRNA on an agarose gel and transferring the mRNA from the gel to a membrane, such as nitrocellulose. In another format, the probes are immobilized on a surface and the mRNA (or cDNA) is contacted with the probes, for example, in a gene chip array. A skilled artisan can adapt known mRNA detection methods for detecting the level of an mRNA.

The level of mRNA (or cDNA prepared from it) in a sample encoded by a gene to be examined can be evaluated with nucleic acid amplification, e.g., by standard PCR (U.S. Pat. No. 4,683,202), RT-PCR (Bustin S. J Mol Endocrinol. 25:169-93, 2000), quantitative PCR (Ong Y. et al., Hematology. 7:59-67, 2002), real time PCR (Ginzinger D. Exp Hematol. 30:503-12, 2002), and in situ PCR (Thaker V. Methods Mol Biol. 115:379-402, 1999), or any other nucleic acid amplification method, followed by the detection of the amplified molecules using techniques known in the art.

The term “primer” refers to any nucleic acid that is capable of hybridizing at its 3′ end to a complementary nucleic acid molecule, and that provides a free 3′ hydroxyl terminus which can be extended by a nucleic acid polymerase. As used herein, amplification primers are a pair of nucleic acid molecules that can anneal to 5′ or 3′ regions of a gene (plus and minus strands, respectively, or vice-versa) and contain a short region in between. Under appropriate conditions and with appropriate reagents, such primers permit the amplification of a nucleic acid molecule having the nucleotide sequence flanked by the primers. For in situ methods, a cell or tissue sample can be prepared and immobilized on a support, such as a glass slide, and then contacted with a probe that can hybridize to mRNA. Alternative methods for amplifying nucleic acids corresponding to expressed RNA samples include those described in, e.g., U.S. Pat. No. 7,897,750.

In another embodiment, the methods of the invention further include contacting a control sample with a compound or agent capable of detecting the mRNA of a gene and comparing the presence of the mRNA in the control sample with the presence of the RNA in the test sample.

The above-described nucleic acid-based diagnostic methods can provide qualitative and quantitative information to determine whether a subject has or is predisposed to a disease characterized by cancer, the potential for the cancer to metastasize, or the metastasis of the cancer.

A variety of methods can be used to determine the level of the polypeptide encoded by a gene disclosed herein. In general, these methods include contacting an agent that selectively binds to the polypeptide, such as an antibody, to evaluate the level of polypeptide in a sample. Antibodies can be polyclonal, or more preferably, monoclonal. An intact antibody, or a fragment thereof (e.g., Fab or F(ab′)2) can also be used. In a preferred embodiment, the antibody bears a detectable label. The FRY antibody described herein that binds to SEQ ID NO. 3 is one such example. The term “label” refers to a composition detectable by spectroscopic, photochemical, biochemical, immunochemical, chemical, or other physical means. For example, useful labels include ³²P, fluorescent dyes, electron-dense reagents, enzymes (e.g., as commonly used in an ELISA), biotin, digoxigenin, or haptens and other entities which can be made detectable. A label may be incorporated into nucleic acids and proteins at any position. The term “labeled”, with regard to the probe or antibody, is intended to encompass direct labeling of the probe or antibody by physically linking a detectable substance to the probe or antibody, as well as indirect labeling of the probe or antibody by reactivity with a detectable substance. For example, an antibody with a rabbit Fc region can be indirectly labeled using a second antibody directed against the rabbit Fc region, wherein the second antibody is coupled to a detectable substance. Examples of detectable substances are provided herein. Appropriate detectable substance or labels include radio isotopes (e.g., ¹²⁵I, ¹³¹I, ³⁵S, ³H, or ³²P), enzymes (e.g., alkaline phosphatase, horseradish peroxidase, luciferase, or β-glactosidase), fluorescent moieties or proteins (e.g., fluorescein, rhodamine, phycoerythrin, Green Flourescent Protein (GFP), or Blue Fluorescent Protein (BFP)), or luminescent moieties (e.g., Qdot™ nanoparticles by the Quantum Dot Corporation, Palo Alto, Calif.).

The detection methods can be used to detect a polypeptide in a biological sample in vitro as well as in vivo. In vitro techniques for detection of the polypeptide include ELISAs, immunoprecipitations, immunofluorescence, EIA, RIA, and Western blotting analysis. In vivo techniques for detection of the polypeptide include introducing into a subject a labeled anti-antibody. For example, the antibody can be labeled with a detectable substance as described above. The presence and location of the detectable substance in a subject can be detected by standard imaging techniques.

The diagnostic methods described herein can identify subjects having, or at risk of developing cancer. The prognostic assays described herein can be used to determine whether a subject is suitable to be administered with an agent (e.g., an agonist, antagonist, peptidomimetic, protein, peptide, nucleic acid, small molecule, or other drug candidate) to treat cancer. For example, such assays can be used to determine whether a subject can be administered with the pharmaceutical compositions described above or other suitable agents to treat the cancer.

Another aspect of the method for identifying a subject at risk of developing cancer involves detecting a susceptibility polymorphism in the FRY gene of a subject, wherein the presence of a susceptibility polymorphism is predictive of increased risk for developing cancer. A susceptibility polymorphism in the FRY gene is a polymorphism that confers loss of function of the FRY polypeptide or absence of the FRY polypeptide. Loss of function is defined as loss of ability to suppress tumor growth and/or induce differentiation. Assays to determine loss of FRY function are known in the art and described hereinbelow. In one embodiment, the polymorphism is a single nucleotide polymorphism that results in an amino acid substitution in a conserved region of the FRY polypeptide. Susceptibility polymorphisms may be detected by methods known in the art, for example, by genotyping by sequencing or hybridization. In cases in which the polymorphism is in a coding region and results in an amino acid change, analysis of amino acid variation in the polypeptide may be used to detect the polymorphism.

Also provided by this invention is a method of monitoring a treatment or determining the effectiveness of the treatment for cancer in a subject. For this purpose, gene expression levels of the genes disclosed herein can be determined for test samples from a subject before, during, or after undergoing a treatment. The magnitudes of the changes in the levels as compared to a baseline level are then assessed. In general a sample of the cancer is obtained prior to treatment, a sample is obtained following the treatment, the level of FRY is compared between the two samples, and one with ordinary skill in the art can further determine whether the treatment is effective according to whether the level of the FRY polypeptide increased in the post treatment sample. An increase in the level of the FRY polypeptide indicates that the treatment is effective, and a medical practioner can further determine the appropriate therapy, dosage and regiment for any follow-up treatment.

### 4. FRY Antibody and Kits Containing FRY Antibodies and Polynucleotides

In another aspect, the present invention provides FRY antibodies and kits embodying the methods, compositions, and systems for the analysis of FRY gene expression and detection of FRY polypeptides in a biological sample as described herein.

Provided herein are isolated antibodies that bind to the FRY polypeptide. The antibody of the invention, which is against FRY, can be obtained by immunizing an animal with FRY or an arbitrary polypeptide selected from the amino acid sequence of FRY, or an epitope selected within the FRY polypeptide, and collecting and purifying the antibody produced in vivo according to a common procedure, known to those with skill in the art. The FRY polypeptide contains a peptide region WGVRRRSLDSLDKC (SEQ ID NO. 3). Routine methods to produce SEQ ID NO. 3 as an epitope on an antigen are known in the art. In certain embodiments, SEQ ID NO. 3 can be used as an epitope on an antigen and an animal can be immunized with SEQ ID NO. 3. The antigen may comprise an epitope that is substantially identical to SEQ ID NO. 3. The epitope may also be a conservatively modified variant of SEQ ID NO. 3. A monoclonal antibody can be obtained by fusing antibody-producing cells which produce an antibody against FRY with myeloma cells to establish a hybridoma according to a known method (for example, Kohler and Milstein, Nature, (1975) 256, pp. 495-497; Kennet, R. ed., Monoclonal Antibodies, pp. 365-367, Plenum Press, N.Y. (1980)).

Antibodies that bind to FRY or an epitope selected within the FRY polypeptide can be obtained using phage display technology. In a further embodiment, antibodies that bind to FRY and SEQ ID NO. 3 can also be obtained using phage display technology. Examples of phage display methods that can be used to make the antibodies of the present invention include those disclosed in Brinkman et al., J. Immunol. Methods 182:41-50 (1995); Ames et al., J. Immunol. Methods 184:177-186 (1995); Kettleborough et al., Eur. J. Immunol. 24:952-958 (1994). The present invention also includes polynucleotides that encode SEQ ID NO. 3 and vectors that contain the polynucleotides that encode SEQ ID NO. 3.

The antibodies that bind to FRY and SEQ ID NO. 3 may also be detectably labeled. Non-limiting examples of labels include radioactive isotopes, enzymes, enzyme fragments, enzyme substrates, enzyme inhibitors, coenzymes, catalysts, fluorophores, dyes, chemiluminescers, luminescers, or sensitizers; a non-magnetic or magnetic particle, iodinated sugars that are used as radioopaque agents, and can be appended to a linker and conjugated to the antibody. Such methods are known to one with ordinary skill in the art, for example, BIOCONJUGATE TECHNIQUES (Academic Press; 1st edition, Greg T. Hermanson, 1996) describes techniques for modifying or cros slinking of biomolecules.

In a further aspect, the invention provides a kit comprising one or more detection reagents which specifically bind to a FRY polypeptide. Preferably, the kit includes an antibody that binds to (SEQ ID NO:3). The detection reagents may be peptide sequences known to flank the FRY antibodies which bind to the FRY polypeptides. The reagents may be bound to a solid matrix or packaged with reagents for binding them to the matrix. The solid matrix or substrate may be in the form of beads, plates, tubes, dip sticks, strips or biochips. Biochips or plates with addressable locating and discreet microtitre plates are particularly useful.

Detection reagents include wash reagents and reagents capable of detecting bound antibodies (such as labeled secondary antibodies), or reagents capable of reacting with the labeled antibody. The kit will also conveniently include a control reagent (positive and/or negative) and/or a means for detecting the antibody. Instructions for use may also be included with the kit. Most usually, the kits will be formatted for assays known in the art, for example, ELISA assays, as are known in the art.

The kit may be comprised of one or more containers and may also include collection equipment, for example, bottles, bags (such as intravenous fluids bags), vials, syringes, and test tubes. Other components may include needles, diluents and buffers. Usefully, the kit may include at least one container comprising a pharmaceutically-acceptable buffer, such as phosphate-buffered saline, Ringer's solution and dextrose solution.

Antibodies used in the assays and kits may be monoclonal or polyclonal and may be prepared in any mammal, or by other known methods known in the art. Antibody binding studies may be carried out using any known assay method, such as competitive binding assays, non-competitive assays, direct and indirect sandwich assays, fluoroimmunoassays, immunoradiometric assays, luminescence assays, chemiluminesence assays, enzyme linked immunofluorescent assays (ELIFA) and immunoprecipitation assays. Zola, Monoclonal Antibodies: A Manual of Techniques, pp. 147-158 (CRC Press, Inc., 1987); Harlow and Lome (1998) Antibodies, A Laboratory Manual, Cold Spring Harbour Publications, New York.

The present invention provides polynucleotides to identify polynucleotides that encode the FRY polypeptide in a biological sample. Such polynucleotide sequences include SEQ ID NO: 1 and fragments thereof, and the complement of SEQ ID NO:1 and fragments thereof, and sequences that hybridize to the foregoing sequences under high stringency conditions, and the primers disclosed herein. One with ordinary skill in the art using common recombinant methods can determine the polynucleotides to be used to detect a polynucleotide that encodes the FRY polypeptide.

The polynucleotides disclosed herein can be included in a kit to determine the presence of the FRY gene in a biological sample. Such a kit may contain a nucleic acid described herein together with any or all of the following: assay reagents, buffers, probes and/or primers, and sterile saline or another pharmaceutically acceptable emulsion and suspension base. In addition, the kit may include instructional materials containing directions (e.g., protocols) for the practice of the methods described herein. For example, the kit may be a kit for the amplification, detection, identification or quantification of a target FRY mRNA sequence. To that end, the kit may contain a poly(T) primer, a forward primer, a reverse primer, and a probe.

In one example, a kit of the invention includes one or more microarray slides (or alternative microarray format) onto which a plurality of different nucleic acids (each corresponding to different regions or nucleic acid variants of the FRY gene) have been deposited. The kit can also include a plurality of labeled probes. Alternatively, the kit can include a plurality of polynucleotide sequences suitable as probes and a selection of labels suitable for customizing the included polynucleotide sequences, or other polynucleotide sequences at the discretion of the practitioner. Commonly, at least one included polynucleotide sequence corresponds to a control sequence, e.g., a “housekeeping” gene, β-actin or the like. Exemplary labels include, but are not limited to, a fluorophore, a dye, a radiolabel, an enzyme tag, that is linked to a nucleic acid primer.

In one embodiment, kits that are suitable for amplifying nucleic acid corresponding to the expressed RNA samples are provided. Such a kit includes reagents and primers suitable for use in any of the amplification methods described above. Alternatively, or additionally, the kits are suitable for amplifying a signal corresponding to hybridization between a probe and a target nucleic acid sample (e.g., deposited on a microarray).

In addition, one or more materials and/or reagents required for preparing a biological sample for gene expression analysis are optionally included in the kit. Furthermore, optionally included in the kits are one or more enzymes suitable for amplifying nucleic acids, including various polymerases (RT, Taq, etc.), one or more deoxynucleotides, and buffers to provide the necessary reaction mixture for amplification.

Typically, the kits are employed for analyzing gene expression patterns using mRNA as the starting template. The mRNA template may be presented as either total cellular RNA or isolated mRNA; both types of sample yield comparable results. In other embodiments, the methods and kits described in the present invention allow quantitation of other products of gene expression, including tRNA, rRNA, or other transcription products.

Optionally, the kits of the invention further include software to expedite the generation, analysis and/or storage of data, and to facilitate access to databases. The software includes logical instructions, instructions sets, or suitable computer programs that can be used in the collection, storage and/or analysis of the data. Comparative and relational analysis of the data is possible using the software provided.

### 5. Methods to Screen for Compounds that Induce the Expression of FRY

The present invention provides in vitro and in vivo methods for screening for candidate compounds for the treatment of cancer, comprising the determination of the ability of a compound to increase the expression or the activity of the FRY polypeptide or the expression of the FRY gene thereof or the activity of at least one of the promoters thereof, said modulation indicating the usefulness of the compound for the treatment of cancer. The method therefore makes it possible to select compounds capable of increasing the expression or the activity of FRY, or the expression of the gene thereof, or the activity of at least one of the promoters thereof.

More particularly, this aspect of the invention is an in vitro method for screening for candidate compounds for the treatment of cancer, comprising the following steps:

a. preparing at least two biological samples or reaction mixtures;

b. bringing one of the samples or reaction mixtures into contact with one or more of the test compounds;

c. measuring the expression or the activity of the FRY polypeptide, the expression of the gene thereof or the activity of at least one of the promoters thereof, in the biological samples or reaction mixtures;

d. selecting the compounds for which an increase of the expression or of the activity of the FRY polypeptide, of the expression of the gene thereof or of the activity of at least one of the promoters thereof, is measured in the sample or the mixture treated in b), compared with the untreated sample or with the untreated mixture.

A “reaction mixture” includes all the necessary reagents and biological materials that replicate the activity or expression of a FRY polypeptide, as known to one with ordinary skill in the art.

An in vivo screening method can be carried out in any laboratory animal, for example, a rodent. According to one preferred embodiment, the screening method comprises administering the test compound to the animal, then optionally sacrificing the animal by euthanasia, and taking a sample of the tumor, before evaluating the expression of the gene in the tumor, by any method described herein.

The compounds tested may be of any type. They may be of natural origin or may have been produced by chemical synthesis. This may involve a library of structurally defined chemical compounds, uncharacterized compounds or substances, or a mixture of compounds. Examples of compounds included but are not limited to small molecules, polypeptides, nucleic acids, carbohydrates or any combination thereof.

Various techniques can be used to test these compounds and to identify the compounds of therapeutic interest which increase the expression or the activity of the FRY polypeptide. According to a first embodiment, the biological samples are cells transfected with a reporter gene functionally linked to all or part of the promoter of the gene encoding the FRY polypeptide, and step c) described above comprises measuring the expression of said reporter gene.

The reporter gene may in particular encode an enzyme which, in the presence of a given substrate, results in the formation of colored products, such as CAT (chloramphenicol acetyltransferase), GAL (beta-galactosidase) or GUS (beta-glucuronidase). It may also be the luceriferase gene or the GFP (green fluorescent protein) gene. The assaying of the protein encoded by the reporter gene, or of the activity thereof, is carried out conventionally by colorimetric, fluorometric or chemiluminescence techniques, inter alia.

According to a second embodiment, the biological samples are cells expressing the gene encoding the FRY polypeptide, and step c) described above comprises measuring the expression of said gene.

The cells used herein may be of any type. It may be a cell expressing the FRY gene endogenously, for instance a normal differentiated cell from breast tissue. Cells that lack the expression or expresses a low level of the FRY polypeptide, for example a triple negative breast cancer cell may be used. Other types of cells include cells that possess a stem cell phenotype.

It may also be a cell transformed with a heterologous nucleic acid encoding the preferably human, or mammalian, FRY polypeptide. A large variety of host-cell systems may be used, such as, for example, Cos-7, CHO, BHK, 3T3 or HEK293 cells. The nucleic acid may be transfected stably or transiently, by any method known to those skilled in the art, for example by calcium phosphate, DEAE-dextran, liposome, virus, electroporation or microinjection.

In these methods, the expression of the FRY gene or of the reporter gene can be determined by evaluating the level of transcription of said gene, or the level of translation thereof.

The expression “level of transcription of a gene” is intended to mean the amount of corresponding mRNA produced. The expression “level of translation of a gene” is intended to mean the amount of protein produced. Those skilled in the art are familiar with the techniques for quantitatively or semi-quantitatively detecting the mRNA of a gene of interest. Techniques based on hybridization of the mRNA with specific nucleotide probes are the most common (Northern blotting, RT-PCR (reverse transcriptase polymerase chain reaction), quantitative RT-PCR (qRT-PCR), RNase protection). It may be advantageous to use detection labels, such as fluorescent, radioactive or enzymatic agents or other ligands (for example, avidin/biotin).

In particular, the expression of the gene can be measured by real-time PCR or by RNase protection. The term “RNase protection” is intended to mean the detection of a known mRNA among the poly(A)-RNAs of a tissue, which can be carried out using specific hybridization with a labeled probe. The probe is a labeled (radioactive) RNA complementary to the messenger to be sought. It can be constructed from a known mRNA, the cDNA of which, after RT-PCR, has been cloned into a phage. Poly(A)-RNA from the tissue in which the sequence is to be sought is incubated with this probe under slow hybridization conditions in a liquid medium. RNA:RNA hybrids form between the mRNA sought and the antisense probe. The hybridized medium is then incubated with a mixture of ribonucleases specific for single-stranded RNA, such that only the hybrids formed with the probe can withstand this digestion. The digestion product is then deproteinated and repurified, before being analysed by electrophoresis. The labeled hybrid RNAs are detected by autoradiography.

The level of translation of the gene is evaluated, for example, by immunological assaying of the product of said gene. The FRY antibodies used for this purpose may be of polyclonal or monoclonal type. The immunological assaying can be carried out in solid phase or in homogeneous phase; in one step or in two steps; in a sandwich method or in a competition method, by way of nonlimiting examples. According to one preferred embodiment, the capture antibody is immobilized on a solid phase. By way of nonlimiting examples of a solid phase, use may be made of microplates, in particular polystyrene microplates, or solid particles or beads, or paramagnetic beads.

ELISA assays, radioimmunoassays or any other detection technique can be used to reveal the presence of the antigen/antibody complexes formed.

The compounds selected by means of the screening methods defined herein can subsequently be tested on other in vitro models and/or in vivo models (in animals or humans) for their effects on cancer.

Methods are also disclosed herein to differentiate a cell that expresses a low level of FRY polypeptide by introducing into the cell a polynucleotide encoding a FRY polypeptide or a FRY polypeptide. The FRY polynucleotide can be introduced using a vector, transfection or by electroporation, or other methods known in the art.

### EXAMPLES

The invention now being generally described, it will be more readily understood by reference to the following examples, which are included merely for purposes of illustration of certain aspects and embodiments of the present invention, and are not intended to limit the invention.

### Example 1

### Materials and Methods

Animals:

Copenhagen (Cop), Fischer 344 (F344) and Brown Norway (BN) rats were purchased from Harlan Sprague-Dawley, Inc. (Madison, Wis.). To generate F 1 progeny, F344 females were mated with Cop males. Female (F344 X Cop) F1 progeny were crossed with F344 males to generate the N2 backcross progeny. Breeding, treatments, tumor sites, incidence, latencies, phenotypes and genotypes were tracked by a relational database.

Carcinogen Treatments and Phenotyping

Female N2 and F344 rats received a single intraperitoneal injection (50 mg/kg body weight) of N′-Methyl-N′-Nitrosourea (NMU) (Ash-Stevens, Detroit, Mich.) at 50-55 days of age and scored for mammary carcinomas incidence and latency. NMU (Ash-Stevens, Detroit, Mich.) was prepared as a stock solution of 10 mg/ml in a 0.9% NaCl solution acidified to pH 5.0 with acetic acid. Fresh solutions of NMU were prepared every 30 minutes. Following carcinogen exposure, N2 progeny were maintained on a high fat diet (rat chow supplemented with 30% fat, ad libitum) to promote tumor growth. Rats were palpated for tumors on a weekly basis, and euthanized when moribund or before tumors attained a diameter of 1 cm. Mammary tumors and normal mammary tissue from tumor-bearing rats were collected, frozen in liquid nitrogen and stored at −80° C. In addition, a section of each tumor was fixed in formalin, embedded in paraffin and sections stained with hematoxylin and eosin for histopathological analysis. The number and latency of mammary carcinomas were scored for each animal.

Genotyping

Genomic DNAs were isolated from tissues using the QIAGEN DNeasy 96 Tissue Kit (QIAGEN Inc. Valencia, Calif.). Informative Simple Tandem Repeat (STR) markers were selected from Rat Genome Database (http://rgd.mcw.edu/). Swept radii calculated using the Haldane mapping functions were used to estimate the number of markers and N2 backcross progeny required for coverage of the genome at the 95% confidence limits. For low-resolution mapping, 77 polymorphic markers were used to genotype 99 female N2 backcross progeny, phenotyped for susceptibility to NMUinduced mammary carcinogenesis. For high-resolution mapping (1-2 cM), informative markers within intervals yielding significant or suggestive linkage scores in the low-resolution mapping were selected.

Linkage Statistical Analyses

Linkage analysis using tumor number as the quantitative trait was performed using MapManager QTX (K. F. Manly et. Al., Mamm Genome 12, 930, December, 2001). Interactions and association were tested by two-way ANOVA, x² tests, and Logistic analysis models using SAS v.8.0 statistical software package (SAS Institute, Inc).

Fluorescence In-Situ Hybridization (FISH).

Embryonic fibroblasts from Cop X F344 F1 rats and from the BN/SsNHsdMcw strain were cultured, blocked in mitosis with colcemid, and used to prepare metaphase spreads. Rat BAC clones encompassing particular STR markers were purchased from Children's Hospital Oakland Research Institute (CHORI-230 BAC library) and verified by PCR amplification to contain the STR sequence. BAC DNA was isolated on an Autogen 740 system, biotinylated by nick-translation, hybridized to metaphase spreads in the presence of excess unlabeled rat Cotl DNA, and detected with avidin-FITC (Trask 1999). The chromosomes were QFH-banded by DAPI staining. DAPI and FITC images were collected separately, but in conjunction. Hybridization signals were analyzed in at least five, and more typically 10 metaphase cells per probe.

Isolation of Genomic DNA and Genotyping.

PCR primers for STR markers were purchased from Research Genetics, Inc. (Madison, Wis.). The PCR-based genotyping assays were performed using one of two methods. If the lengths of the STRs differed by eight or more base pairs between the F344 and Cop alleles, the PCR reactions were performed with the unlabeled primers purchased from Research Genetics, and the genotypes were determined by amplicon length using polyacrylamide gel electrophoresis (PAGE) analysis. For STRs differing by six or less base pairs, PCR reactions were performed with the fluorescence labeled primers, and the genotypes were determined on an ABI PRISM® 3100 Genetic Analyzer system and Genescan software. Genotype calls were made with Genotyper software (ABI). All the allele calls, including those from regular PAGE analysis, were independently verified by at least one other researcher before importing into the database for further analysis.

PCR Amplification and Sequencing of the Candidate Mcs Genes.

PCR primers for amplification of selected candidate genes were designed to generate two or more overlapping amplicons. RNA was extraction from normal mammary gland using a Qiagen RNeasy maxi kit (Qiagen Inc. Valencia, Calif.) cDNA was produced using reverse transcription reaction according to SuperScript™ II Reverse Transcriptase instruction (Invitrogen, Carlsbad, Calif.). The amplified PCR products were separated by agarose gel electrophoresis (0.8-1.5%), excised from the gel, and purified with Qiagen gel extraction kits (Qiagen Inc. Valencia, Calif.).

DNA sequencing was performed using Big Dye Cycle Sequencing Protocol (PerkinElmer Biosystems Inc. Boston, Mass.). Each PCR product was sequenced on both strands using the same primers used in PCR amplification reaction. The labeled sequencing reaction products were separated on an automated, fluorescence-based sequencer (Model 377 from Applied Biosystems, Foster City, Calif.). Automated base calls were reviewed by visual inspection. Sequences were assembled using Sequencer 4.2 software (Gene Codes Corporation, Ann Arbor, Mich.).

Construction of Rat FRY-GFP Tagged Protein Expression Vector.

The 3′ and 5′ RACE were used to determine the full-length transcript of the rat FRY gene. Amplifications were carried out with the Marathon cDNA Amplification Kit (BDClontech, Palo Alto, Calif.) using two gene-specific primers. Double-stranded cDNA was prepared using Marathon cDNA Amplification Kit, amplified, cloned using the TOPO TA kit, and subjected to DNA sequencing. The 10,791 base pair FRY cDNA was cloned in four sections and then reconstructed in the expression vector. Briefly, the COP rat FRY cDNA (GenBank accession number EU563851.1) was PCR-amplified with primers set 1, 5′-CAGGCATTGCTGCTTATG-3′ and 5′-TCCAAGAACAACGCTCCA-3′; Primer set 2, 5′-CTGGAGAGCATCGAAATC-3′ and 5′-CAAGGCCATCAGGTATTC-3′; Primer set 3,5′-AGCACTGTGACAACCCAC-3′ and 5′-CAGAGCAGGAGGTAAGCA-3′ and Primer set 4,5′-TTGGGAGACGGTATGATG-3′ and 5′-TATAGGATCCGGAGCCTCCTGTCCGAGAC-3′, in which C-end primer was modified by changing the stop codon to a Glycine, and adding a BamHI cloning sites at the end of primer. The overlapping four PCR products were cloned into pCR2.1-TOPO vector following the manufacture's protocol (Invitrogen, Carlsbad, Calif.), and sequenced to confirm that the vectors contains the correct sequence of COP rat FRY gene. The resulting plasmids were named as pCR2.1-1, pCR2.1-2, pCR2.1-3 and pCR2.1-4, respectively. The pCR2.1-1 and empty pEGFP-N1 expression vectors were excised with Xho I/BamH I restriction enzymes (Clontech, Mountain View, Calif.) (NEB, Ipswich, Mass.), and the products were gel purified using the QlAquick Gel Extraction kit (Qiagen, Valencia, Calif.) and ligated together using T4 DNA ligase (Invitrogen, Carlsbad, Calif.). The resulting plasmid was sequenced and named pEGFP-N1-1 containing the PCR product amplified by the first set of primers. Following the same procedure, the Xba I/BamH I restriction enzymes digested pCR2.1-2 and pEGFP-N1-1 vectors were used to generate pEGFP-N 1-2. The pEGFP-N 1-3 plasmid was generated using pEGFP-N 1-2 and pCR2.1-3, which were excised by Not I/BamH I restriction enzymes. Finally, the pEGFPN1-COP plasmid containing the whole coding sequence of COP rat FRY gene was produced from pEGFP-N1-3 and pCR2.1-4 vectors, excised by EcoR V/BamH I restriction enzymes.

Cell Culture:

Cell cultures were maintained at 37° C. in a humidified atmosphere of 5% CO2 and 95% air. MCF-10A and 10A-shFRY cells were cultured in Mammary Epithelium Basal Medium (MEBM), supplemented with MEGM SingleQuots (Lonza) (gentomicin sulfate amphotericin-B, 20 ng/ml human epidermal growth factor, 10 μg/ml insulin, 5 μg/ml hydrocortisone and bovine pituitary extract). MDA-MB-231 and 231wCFRY cells were cultured in Advanced DMEM with 10% heat inactivated FBS with 100 μg/ml penicillinstreptomycin solution. MCF 10A cells were cultured in MEBM, supplemented with MEGM SingleQuots (Lonza).

Northern Blot Analysis.

Total RNA was extracted from using a Qiagen RNeasy maxi kit (Qiagen Inc. Valencia, Calif.). RNA samples (30 μg) were separated by electrophoresis through a 0.8% (w/v) formaldehyde/agarose gel, transferred to Hybond-N nylon membranes (Amersham Pharmacia Biotech), and probed with an [a-32P]-dCTP labeled probes. Following hybridization and washing, autoradiography was performed by exposure to X-ray film.

Semiquantitative (RT)-PCR.

Total RNA was isolated from tissues and cell lines using the Qiagen RNEasy Mini kit (Qiagen, Valencia, Calif.). Reverse transcription was performed with the SuperScript™ II Reverse Transcriptase kit (Invitrogen, Carlsbad, Calif.). PCR conditions for DNA amplification in the linear range were established on a GeneAmp PCR System 7600 (PerkinElmer, inc. Wellesley, Mass.). Platinum® Taq DNA Polymerase (Invitrogen, Carlsbad, Calif.) and primers were used for DNA amplification. RT-PCR products were analyzed on 0.9% agarose gels and expression was normalized to β-actin.

SNAP Analysis.

The SNAP (Screening for Non-Acceptable Polymorphisms) computational tool predicts the functional consequences of single amino acid substitutions in both binary (neutral/non-neutral, with respect to wild type function) and scored form (−100 to +100, where negative predictions are neutral, positive are non-neutral, and higher absolute values of scores indicate higher reliability of the binary prediction). SNAP was used to indicate whether the non-synonymous SNPs in the Fisher 344 rat strain are indicative of functional effects. The SNAP output scores were evaluated for each amino acid substitution at the codons in question. SNAP also provides the likely functional (as opposed to structural) importance of each amino acid in the protein sequence by computing the SNAP-BLOSUMB62 score—for each wild-type residue, the average SNAP score of substitutions allowed by the BLOSUM62-matrix at cutoff ≧0. As with regular SNAP scores, SNAP-BLOSUM62 scores ≦0 indicate that a specific sequence position is not likely functionally significant, and a score >0 indicates that this location is probably functionally significant.

Nude Mouse Tumorigenicity Assays.

Athymic nude mice (NCR-NU) were purchased from Taconic. Animal subjects were housed in groups of three, and maintained on a 12-hour light/dark cycle with food and water ad libidum. In vivo tumorigenicity of cell lines was assessed by subcutaneous injection of 2.5×10⁶ viable cells, in a maximum volume of 100 μl, into the intrascapular region of male nude mice using a tuberculin syringe with a 26-gauge needle. Cells were prepared by trypsin digestion, counted, tested for viability with trypan blue vital staining and suspended in Hank's balanced salt solution. To ensure that the proposed number of animals per experiment is adequate to test the hypotheses of interest, six mice were used per cell type, giving us an alpha error level of 5% (or a 95% confidence interval) and power of 0.55 (beta error level of 45%; statistical power=1−beta).

Tumors were measured twice a week using digital calipers. The tumor growth rates and end volumes were compared between the cell lines. On termination of the study, mice were sacrificed using asphyxiation with CO2 followed by cervical dislocation of dead animals, tumor tissues were removed and portions were fixed in 10% formalin and frozen in liquid nitrogen. Portions of each tumor were sectioned and stained for histopathological analysis using: 1) hematoxylin and eosin and 2) masson's trichrome stain. The tumor volumes were estimated using the formulas:

Spherical Tumors: (4/3) pi r3

Ellipsoid Tumors: (4/3) pi (r1) (r2) (r3)

Microarray Analysis.

Forty nanograms of total RNA from each sample were used to generate a high fidelity cDNA for array hybridization. The cDNA were amplified, fragmented and labeled with biotin using NuGEN Ovation Amplification and Labeling kit V2 (NuGEN, San Diego, Calif.). SPIA product and fragmented cDNA are analyzed by electrophoresis using the Agilent Bioanalyzer 2100 to assess the appropriate size distribution prior to microarray hybridization. 3.75 μg of amplified and labeled cDNA was used in the hybridization cocktail for GeneChip analysis. Detailed protocols for sample preparation suing the NuGEN Ovation amplification and labeling kits can be found at http://www.nugeninc.com.

All samples were subjected to gene expression analysis via the Affymetrix Human Genome U 13 3 Plus 2.0 high-density oligonucleotide array that currently queries over 47,000 transcripts. Hybridization, staining and washing of all arrays were performed in the Affymetrix fluidics module as per the manufacturer's protocol. Streptavidin phycroerythrin stain (SAPE, Molecular Probes) was the fluorescent conjugate used to detect hybridized target sequences. The detection and quantification of target hybridization was performed with a GeneArray Scanner 3000 (Affymetrix). All arrays referred to in this study were assessed for “array performance” prior to data analysis.

The raw microarray data was analyzed using GeneMaths (Applied Math). LIMMA (Linear Models for Microarray Analysis) were then utilized to fit a linear model to the expression data (log-ratios) of each gene (Smyth 2005). The false discovery rate was controlled using the Benjamin and Hockberg procedure (Benjamini 1995).

Transfection of MDA-MB-231 with the wild-type Copenhagen FRY allele

The pAcGFP1-N1 (Clontech Cat. No. 632469) plasmid vector containing the wild-type Cop allele of the FRY gene (pAcGFP1-N1-COP) was isolated from bacterial cultures. pAcGFP1-N1 was transfected into subconfluent MDA-MB-231 cells with DoTap Transfection Reagent (Roche) for 6 hours. Cells were then cultured in serum containing medium and allowed to recover for 48 hours. G418 (Sigma) was added at a concentration of 800 μg/ml to select for cells that had incorporated pAcGFP1-N1 in their genomes. Individual G418-resistant colonies were isolated with cloning cylinders. Individual clonal lines were expanded and examined for the expression of both rat and human FRY expression before evaluation in in-vitro and in-vivo cell transformation assays.

Transfection of MCF10A with shRNA Directed Against FRY.

A set of five sequence-verified shRNA lentiviral plasmids (containing the parental vector pLK0.1), each containing a different FRY targeting shRNA sequence (Sigma MISSION shRNA SHCLND-NM_{—}023037). A non-targetting shRNA vector was used as a control (Sigma MISSION Non-target shRNA control vector SHC002). Plasmid vectors containing were isolated and transfected into subconfluent MCF 10A cells using DoTap Transfection Reagent (Roche) for 6 hours. Serum containing medium was then reintroduced and the transfected cells were allowed to recover for 48 hours. Puromycin was added at a concentration of 1 μg/ml to select for cells that had incorporated control of shRNA plasmid in their genomes. Individual puromycin-resistant colonies were isolated with cloning cylinders. Clonal lines were expanded and monitored for persistence of decreased FRY expression before use in in-vitro and in-vivo cell transformation assays.

Western Blots.

Total cellular extracts (30 μg protein) were separated electrophoretically on pre-cast 7.5% Tris-HCl gels (Bio-Rad) and transferred to Immobilon-FL (Millipore, cat# IPFL00010) PVDF membranes. The membranes were incubated with FRY antibody (1:10,000) overnight at 4° C. The blots were then rinsed three times with TTBS for 10 min, 7 min, and 5 min, rinsed briefly with TBS and then incubated with LI-COR anti-rabbit 680 nm secondary antibody for 45 minutes under gentle agitation at 4° C. Blots were developed using the LI-COR Odyssey Infrared Imaging System.

Matrigel 3-Dimensional Growth Assay.

Cells were grown in 3-dimensional culture using the methodology described by Lee et al. (S0). The wells of a 6-well plate were coated with a base layer of Matrigel and cells resuspended in MEBM medium were plated atop the base layer at a density of 2.5×10⁴ cells/cm², and allowed to set for 30 min at 37° C. A 10% solution of Matrigel in the appropriate cell culture medium was next applied to the top of the culture. Cells were allowed to grow for 5 days prior to visual evaluation.

Statistical Analysis of FRY mRNA Expression In Human Clinical Cancer Cohorts.

Microarray data available in the Oncomine 3.0 Cancer Profiling Database (http://www.oncomine.org) were checked for normality using the D'Agostino & Pearson omnibus normality test. If the datasets passed the normality test, then parametric tests were utilized (Student's t-test (unpaired) or one-way ANOVA) for analysis. Additionally, if either or both of the datasets being analyzed were not normal distributed, then nonparametric tests were utilized (Mann-Whitney or Kruskal-Wallis) for analysis. Gene expression correlations were calculated using the Pearson correlation coefficient with a 95% confidence interval and two-tailed p-value. GraphPad Prism 5.0 software was used to perform all relevant statistical operations.

Ingenuity® Pathway Analysis.

Microarray data were interpreted using Ingenuity Pathway Analysis (IPA) (Ingenuity® Systems, www.ingenuity.com). For the determination of overlapping genes and creation of the Venn diagram a cutoff of p<0.05 to include all significantly changed genes in the 10A-shFRY/10A-CV analysis (2,188 genes) and for the 231wCFRY/MDA-MB-231 analysis (4,896 genes).

Immunohistochemistry.

Anti-FRY (rabbit polyclonal) was optimized on normal human breast tissue slides using Ventana Medical Systems Discovery XT automated immunostainer. Human breast tumor tissue array (TMA) slides (US Biomax, Inc) were deparaffinized and antigen retrieval was performed using CC 1 (Cell Conditioning Solution, Ventana Medical Systems, Cat #950-124) with extended time of 72 minutes. TMA slides were stained for FRY in triplicate and two additional control slides were stained using the pre-immune rabbit serum (pre-serum) or only secondary antibody. AntiFRY antibody and preserum were applied at dilution of 1:2500, and slides were incubated at 37° C. for 1 hour. Ventana′ Universal Secondary antibody (cat #760-4205) was incubated for 1 hour followed by chromogenic detection kit DABMap (Ventana Medical Systems, Cat #760-124). Slides were counterstained with Hematoxylin.

Computer quantitative image analysis of immunohistochemical staining was conducted. The specimens were digitized with a Zeiss/Trestle MedMicro whole slide imaging system under 40× volume scan configurations. Image registration algorithm was applied to each whole slide image to recover the TMA structure and each core images were extracted at 20× equivalent resolution. The core images were color decomposed to isolate DAB signal from hematoxylin counter stain and analyzed to generate Integrated Staining Intensity (ISI), Effective Staining Area (ESA) and Effective Staining Intensity (ESI). The breast tumor tissue array, BR208 (US Biomax, Inc), consists of 60 cases of breast carcinoma and 9 cases of normal breast tissue. Cores were present in triplicate on the breast TMA, BR208 (US Biomax, Inc), and pre-serum staining values were subtracted from the FRY staining values for each core and the log 2 was taken of this result for each core. Values were averaged among patients on each slide and across slides (n=9) for each case. These resulting values were statistically graphed and analyzed.

Cores were also rated by a pathologist, using a numbering system (0: no staining, 1: 0-10% of cells stained, 2: 10-40% of cells stained, 3: 40-70% of cells stained, 4: 70-100% of cells stained). The scores were then averaged per patient (n=3) and the resultant values were statistically analyzed using the students t-test to determine if the means between the groups in question were significantly different.

### Example 2

### QTL Mapping and Comparative Sequence Analyses Identified FRY is a Candidate MCS Gene

Genetic linkage in 324 female [((F344 X Cop) F1 X F344)N2] progeny, using the number of mammary tumors induced by N′-Methyl-N′-Nitrosourea (NMU)-induced mammary carcinogenesis within the first 200 days after exposure as the quantitative trait, identified a new Mcs locus on rat chromosome 12 (RNO12). The locus candidate Mcs showed highly significant linkage to the D12Rat59 simple tandem repeat marker (LOD=8.6); linkage to this region was further confirmed by interval mapping (Z. Zeng, Genetica 123, 25, Feb. 2005) (Tables 1 and 2). The direction of the additive effect in interval mapping confirmed that the presence of the Cop FRY allele was negatively correlated with susceptibility indicating it is a recessive trait. Since the putative Mcs locus was located within a 5.6-centiMorgan region near the centromere on RNO12 whose synteny is conserved on human chromosome 13q12 to 13q13 and includes the rat Brca2, Brca2 was ruled out by DNA sequencing, and by comparing mRNA and protein expression levels between these strains. After eliminating Brca2 and several candidate tumor suppressor genes within this region, a gene located 30 kbp distal to the R12Rat59 marker, and within 2.5 kbp of Brca2 was considered. The gene was subsequently identified as rat FRY, which is highly similar to the human FRY gene (NM_{—}023037), and is an ortholog of the Drosophila furry gene. Rapid Analysis of cDNA Ends (D. J. Park et. Al., Biotechniques 34, 750, April 2003), demonstrated that the rat FRY transcript comprises 10,791 nucleotides, encoding a protein of 3011 amino acids. A putative ATP-binding domain common to the GHMP family of kinases is highly conserved in the FRY polypeptide across species (Table 3), signifying that FRY encodes a protein kinase.

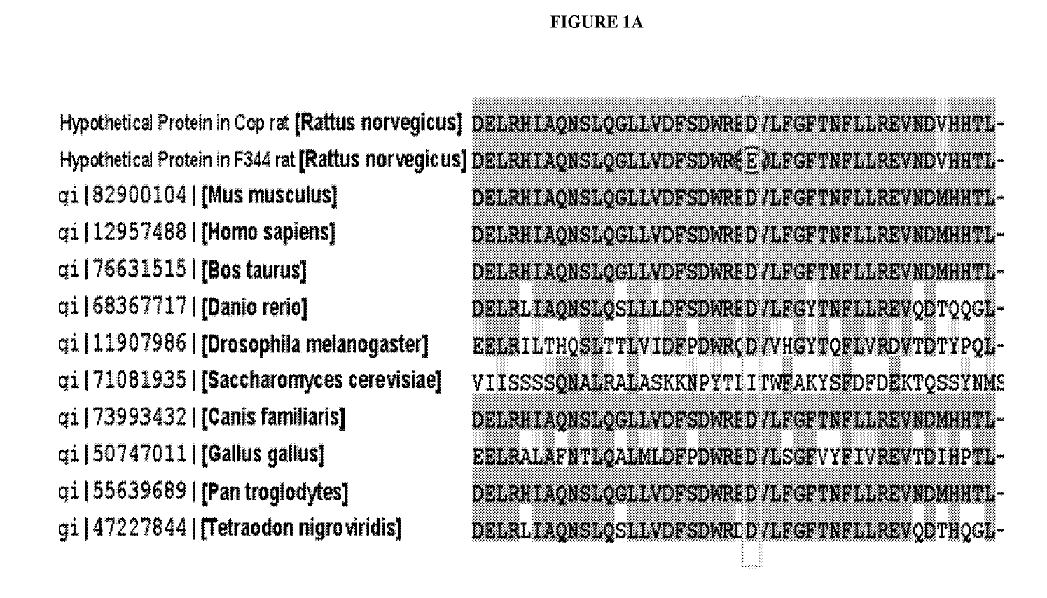

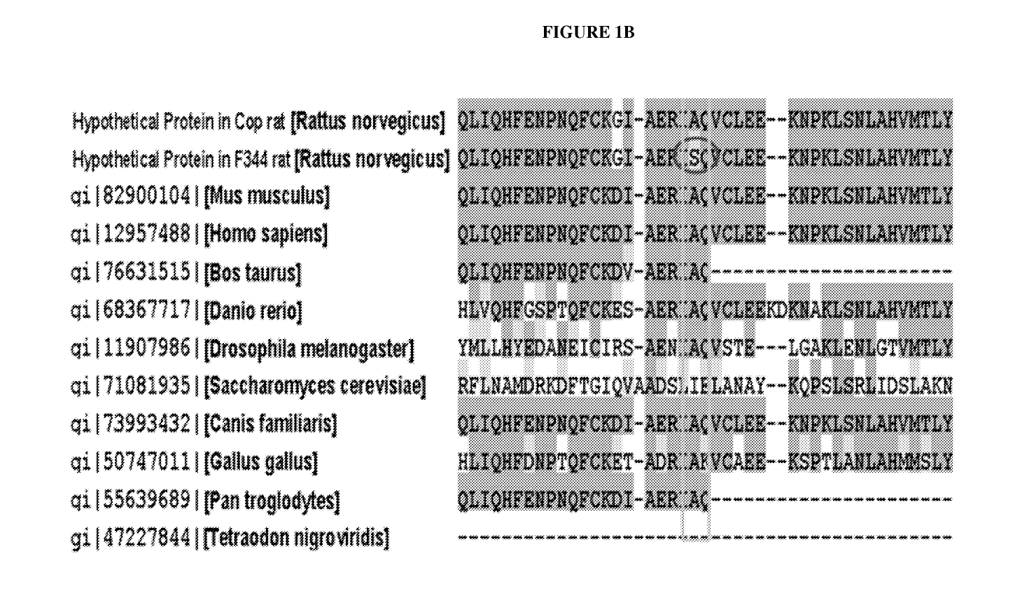

Comparative sequence analysis of FRY among three rat strains (F344, Cop, Brown Norway (BN)), and across multiple species, identified two non-synonymous SNPs that were unique to the FRY gene of the susceptible F344 strain. Both SNPs induce amino-acid substitutions at residues that are highly conserved across evolution (FIG. 1), indicating that these residues may be important for normal FRY function.

The Alanine-to-Serine substitution at amino-acid 2170 creates a de novo phosphorylation consensus sequence for several cancer-related protein kinases (Table 4), and the F344-specific mutation could alter FRY function through aberrant post-translational modification. The SNAP score (Bromberg, B. Rost, Nucleic Acids Res 35, 3823, 2007) for an amino acid substitution at codon 2171 (where the F344 rat had an Alanine to Serine mutation; score+15) indicated that an amino acid change at this location is likely to have functional consequences. The SNAP-BLOSUM62 score indicated that this location is functionally significant (score +8).

TABLE 1
Complete list of Rat STR Markers used for high
and low density linkage analysis.

D1RAT15
D1RAT257
D1RAT266
D1RAT29
D1RAT32
D1RAT183
D1RAT38
D1RAT41
D1RAT272
D1RAT173
D1RAT236
D1RAT49
D1RAT277
D1RAT164
D1RAT282
D1RAT437
D1RAT198
D1RAT65
D1RAT290
D1RAT22
D1RAT181
D1RAT293
D1RAT115
D1RAT76
D1RAT81
D1RAT132
D2RAT10
D2RAT21
D2RAT161
D2RAT38
D2RAT54
D2RAT66
D3RAT17
D3RAT103
D3RAT167
D3RAT220
D3RAT15
D3RAT4
D4RAT115
D4RAT27
D4RAT40
D4RAT6
D4RAT200
D4RAT68
D5RAT17
D5RAT82
D5RAT13
D5RAT149
D5RAT95
D5RAT108
D5RAT35
D5RAT205
D6RAT68
D6RAT135
D6RAT21
D6RAT12
D7RAT31
D7RAT103
D7RAT86
D7RAT100
D7RAT11
D7RAT4
D8RAT51
D8RAT46
D8RAT104
D8RAT65
D9RAT133
D9RAT30
D9RAT18
D9RAT7
D9RAT100
D10RAT182
D10RAT73
D10RAT18
D10RAT8
D11RAT11
D11RAT34
D12RAT3
D12RAT35
D12RAT36
D13RAT70
D13RAT24
D13RAT32
D14RAT5
D14RAT11
D14RAT39
D14RAT94
D15RAT5
D15RAT12
D15RAT71
D16RAT8
D16RAT61
D16RAT34
D17RAT61
D17RAT15
D17RAT32
D18RAT53
D18RAT13
D19RAT15
D19RAT71
D20RAT4
D20RAT5
D20RAT10
D21RAT24
D21RAT111
D21RAT115

TABLE 2
Calculated LOD scores for markers on rat chromosome 12.

| Marker Name | Number of Rats Genotyped | LOD |
|---|---|---|
| D12Rat57 | 324 | 3.3 |
| D12Rat1 | 324 | 6.0 |
| D12Rat59 | 324 | 8.6 |
| D12Arb2 | 324 | 2.1 |
| D12Rat3 | 99 | 2.7 |
| D12Rat35 | 99 | 0.8 |
| D12Rat36 | 99 | 0.5 |

TABLE 3
Occurrence of patterns, profiles and motifs of rat FRY polypeptide.

| Proscansite | | Hit |
|---|---|---|
| Entry | Name of Patterns, profiles and Motifs | Number |
| PS00627 | GHMP kinases putative ATP-binding domain | 1 |
| PS50324 | Serine-rich region profile | 1 |
| PS00006 | Casein kinase II phosphorylation site | 63 |
| PS00005 | Protein kinase C phosphorylation site | 33 |
| PS00001 | N-glycosylation site | 7 |
| PS00007 | Tyrosine kinase phosphorylation site | 3 |
| PS00004 | cAMP- and cGMP-dependent protein kinase | 5 |
| | phosphorylation site | |
| PS00008 | N-myristoylation site | 13 |
| PS00009 | Amidation site | 1 |
| PS00029 | Leucine zipper pattern | 1 |

TABLE 4
Prediction of a novel phosphorylation site in the F344 Fry allele
by NetPhos2.0 Predict software.
NetPhos2.0 Predict Software

| Name | Position | Context | Score | Predicted |
|---|---|---|---|---|
| Sequence | 2170 | AERISQVCL | 0.986 | *S* |

| Site | Kinase | Score |
|---|---|---|
| S-2170 | protein kinase, DNA activated, catalytic polypeptide | 0.51 |
| | (predicted) [Rattus norvegicus] | |
| S-2170 | ataxia telangiectasia mutated homolog (human) (mapped) | 0.55 |
| | [Rattus norvegicus] | |
| S-2170 | protein kinase, cAMP-dependent, catalytic, | 0.60 |
| | [Rattus norvegicus] | |

### Example 3

### Changes in FRY Expression Alters Epithelial Cell Morphology in Vitro

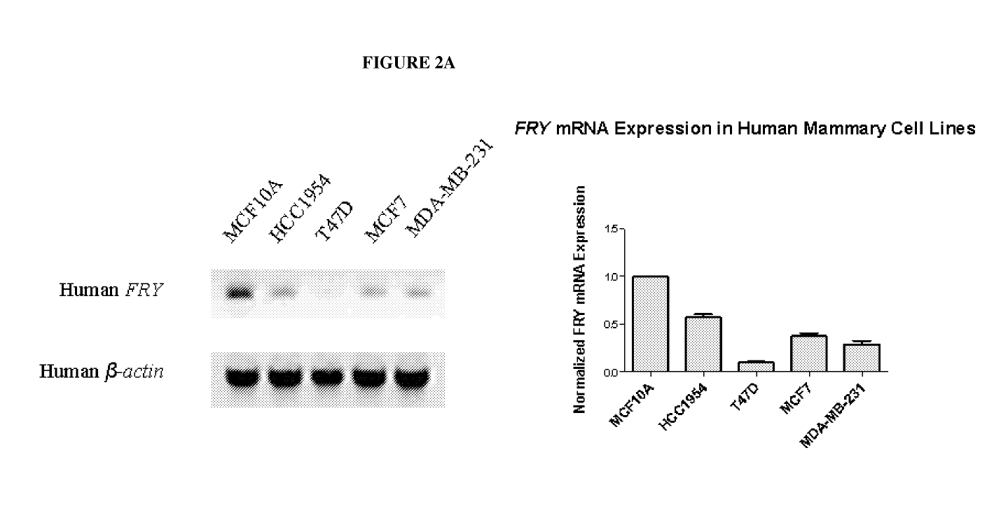

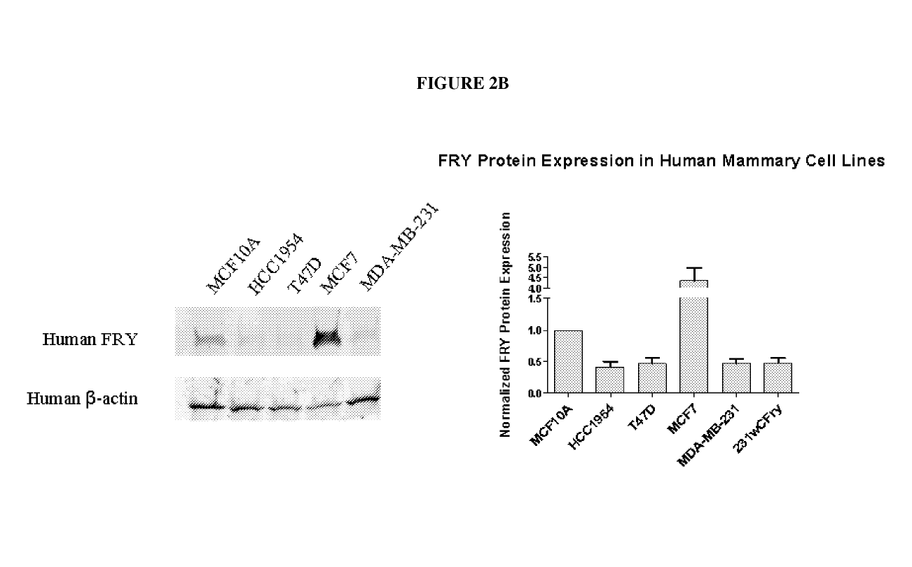

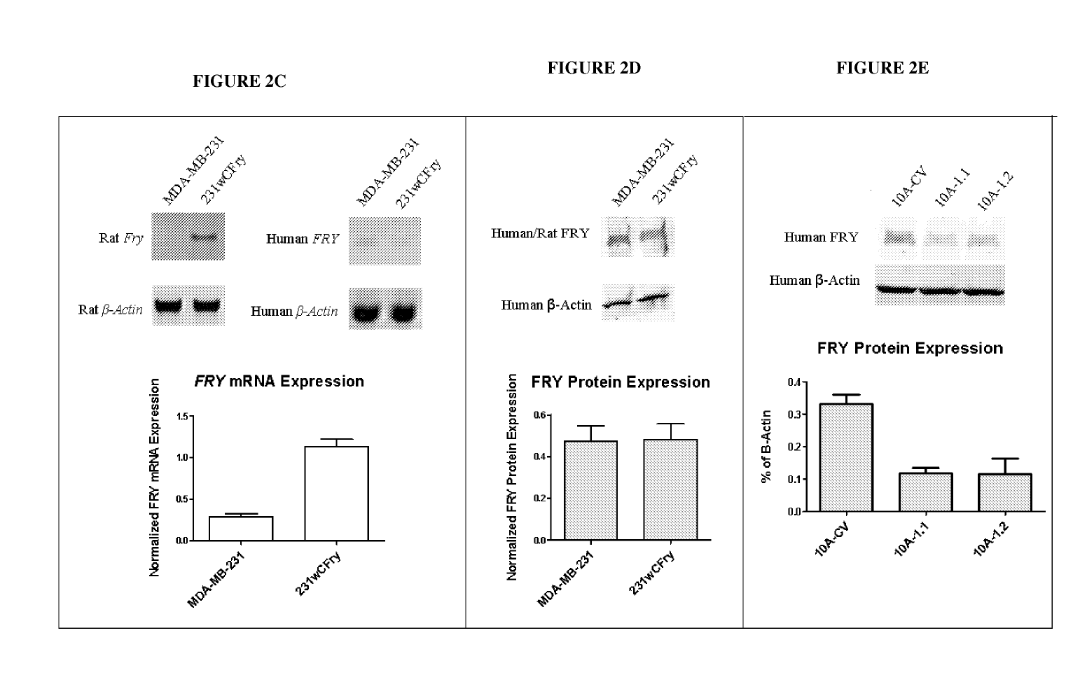

The non-tumorigenic MCF 10A human mammary epithelial cell line, FRY mRNA expression levels were reduced by at least 40% in all breast cancer cell lines tested, and FRY polypeptide expression was decreased in 3 of the 4 breast cancer lines evaluated (FIG. 2 a,b). To test the hypothesis that decreased FRY activity contributes to mammary carcinogenesis, functional analyses using two in vitro models were performed. The wild type Cop FRY allele was expressed in the triple-negative MDA-MB-231 breast cancer cell line (ER−/PR−/HER2−) which was derived from a highly invasive, late-stage mammary epithelial cell tumor and is commonly used as a model for metastatic breast cancer. Several clones were isolated that express FRY at levels similar to the non-tumorigenic MCF 10A cell line for phenotypic analysis, ensuring that any changes in phenotype were unlikely to result from over-expression of the Cop FRY allele (FIG. 2 c,d). Second, shRNA was targeted to the FRY mRNA to knock down expression of the endogenous FRY in the nontumorigenic MCF 10A mammary epithelial cell line. MCF10A clones were selected in which FRY mRNA and protein were decreased by approximately 60 relative to the levels in MCF-10A cells stably transfected with the nontargeting shRNA (10A-CV) (FIG. 2e).

Altered FRY expression dramatically affected the morphology and organization of cells grown in both monolayer and three-dimensional cultures. Whereas the parental MDA-MB-231 cells exhibited an undifferentiated spindle-like growth pattern in a monolayer, FRY transfectants exhibited a more organized, epithelial-like, cobblestone pattern (While the 10A-CV cells exhibited an organized, epithelial-like, cobblestone pattern, the 10A-shFRY cells exhibited an altered morphology when cultured in a monolayer. Additionally, when cultured with an overlay of Matrigel™, the cells expressing ectopic FRY formed polarized mammospheres (Debnath, S. K. et. al., Methods 30, 256, July 2003) resembling those formed by the nontumorigenic MCF 10A cell line. By contrast, the parental MDA-MB-231 and the 10A-shFRY cells grew as more disorganized clusters exhibiting a disorganized morphology and decrease in cell adhesion. These findings indicated that increased FRY expression is associated with altered cell morphology, cell polarization and differentiation in vitro.

### Example 4

### The Fry Gene Suppresses Tumorigenicity in Vivo

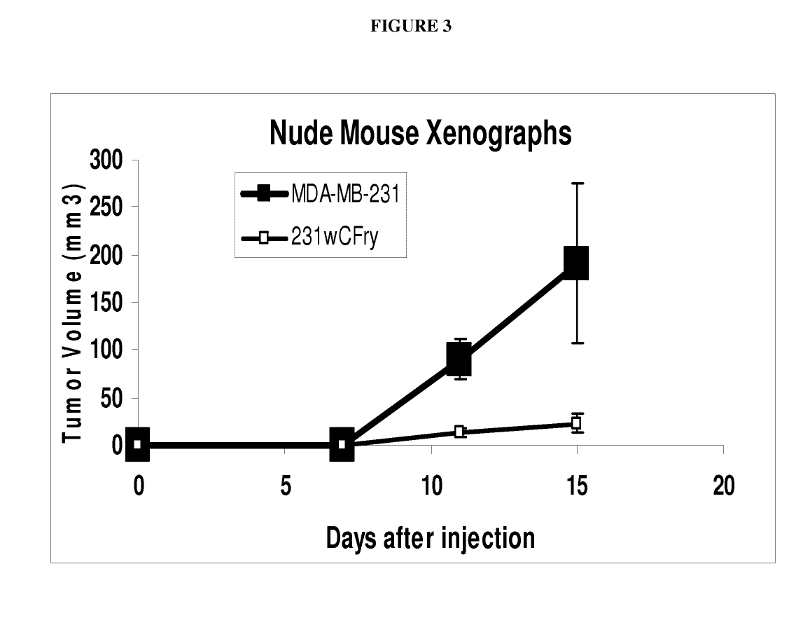

Ectopic FRY expression dramatically reduced tumorigenicity in vivo. Relative to the rapidly growing formed by MDA-MB-231 cells (FIG. 3), subcutaneous nude mice xenografts of the MDA-MB-231 cells ectopically expressing the wt allele of Cop FRY (231wCFRY) showed an ˜8-fold reduction in tumor growth. Additionally, in sharp contrast to the aggressively growing and highly invasive tumors formed by the MDA-MB-231 cells, the small subcutaneous nodules formed by 231wCFRY cells were encapsulated and did not invade the underlying skeletal muscle and/or fat tissue.

### Example 5

### FRY is Decreased in Human Breast Tumors

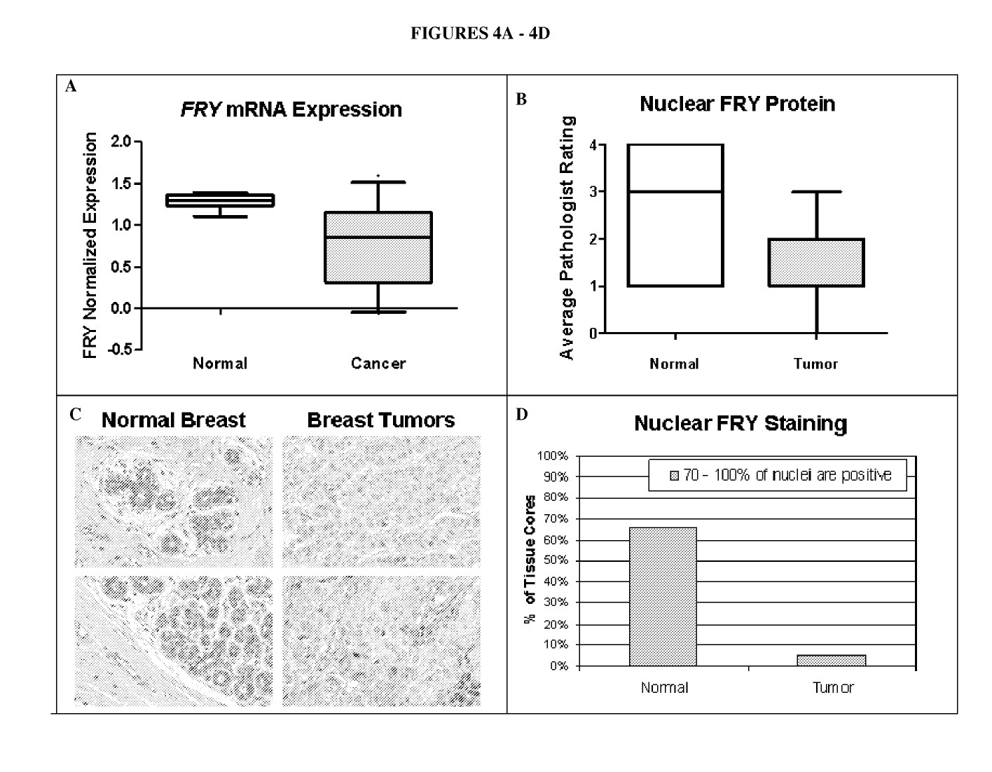

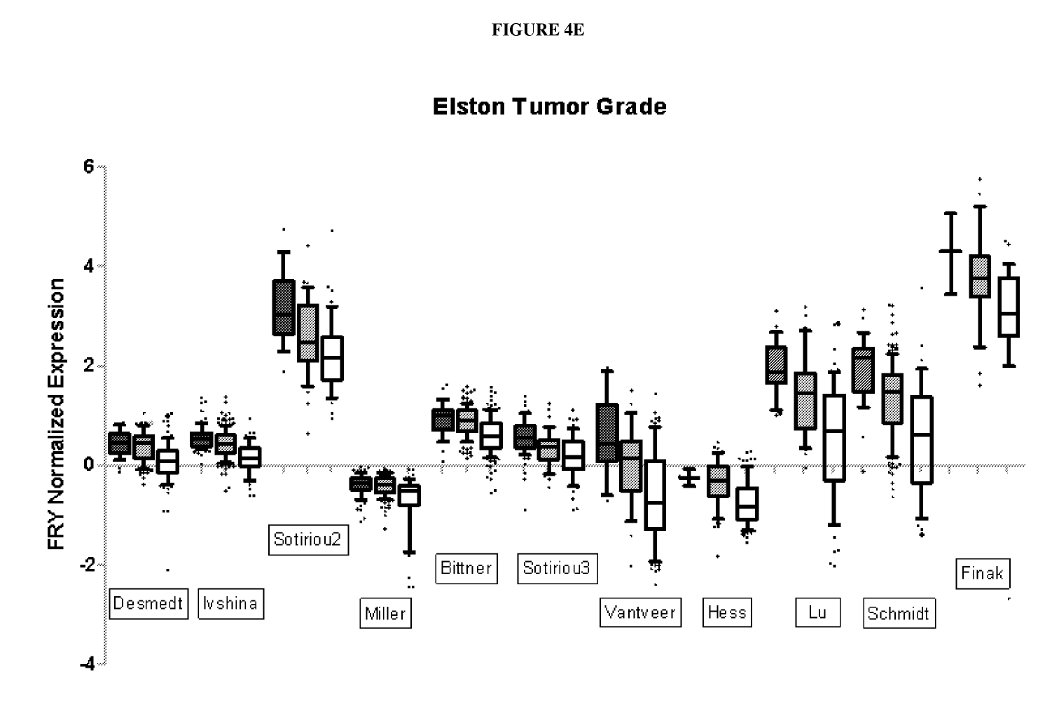

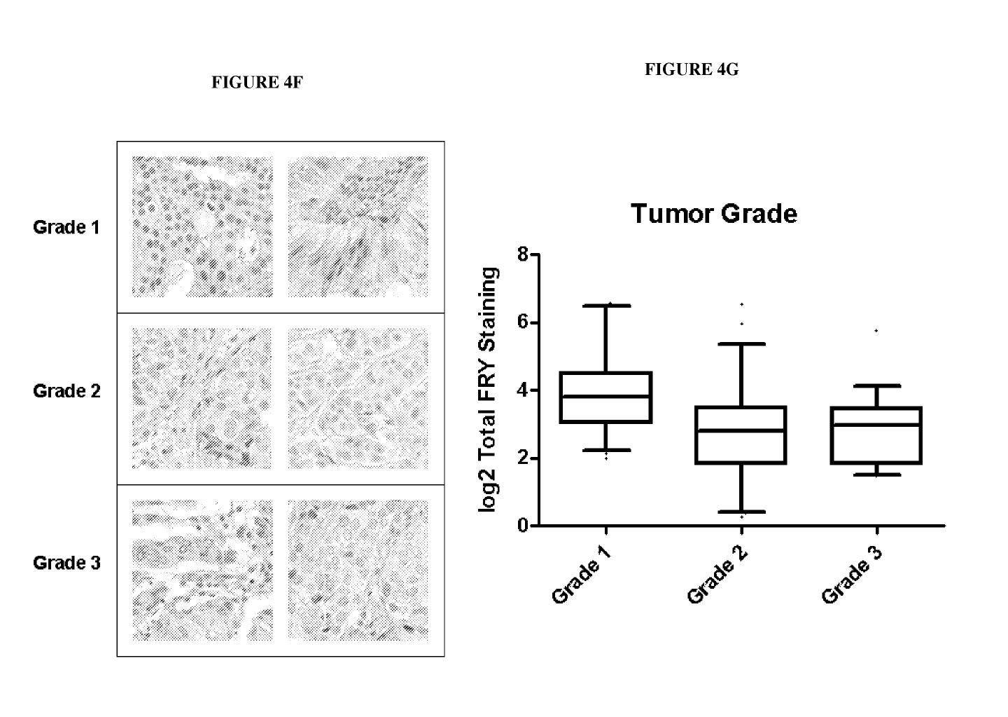

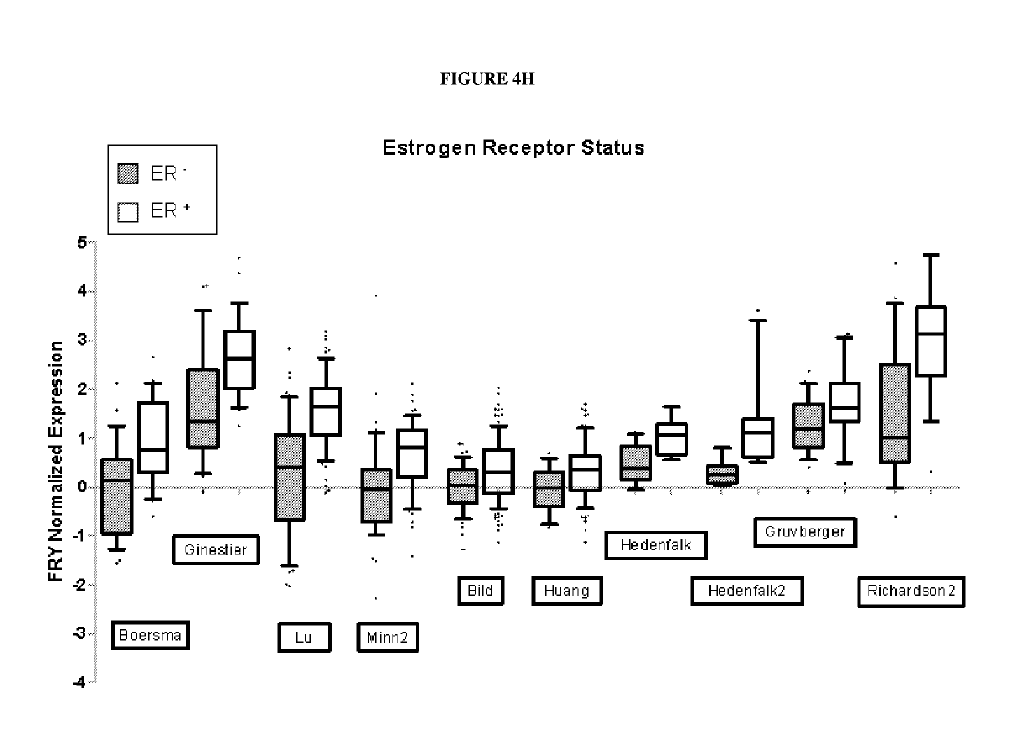

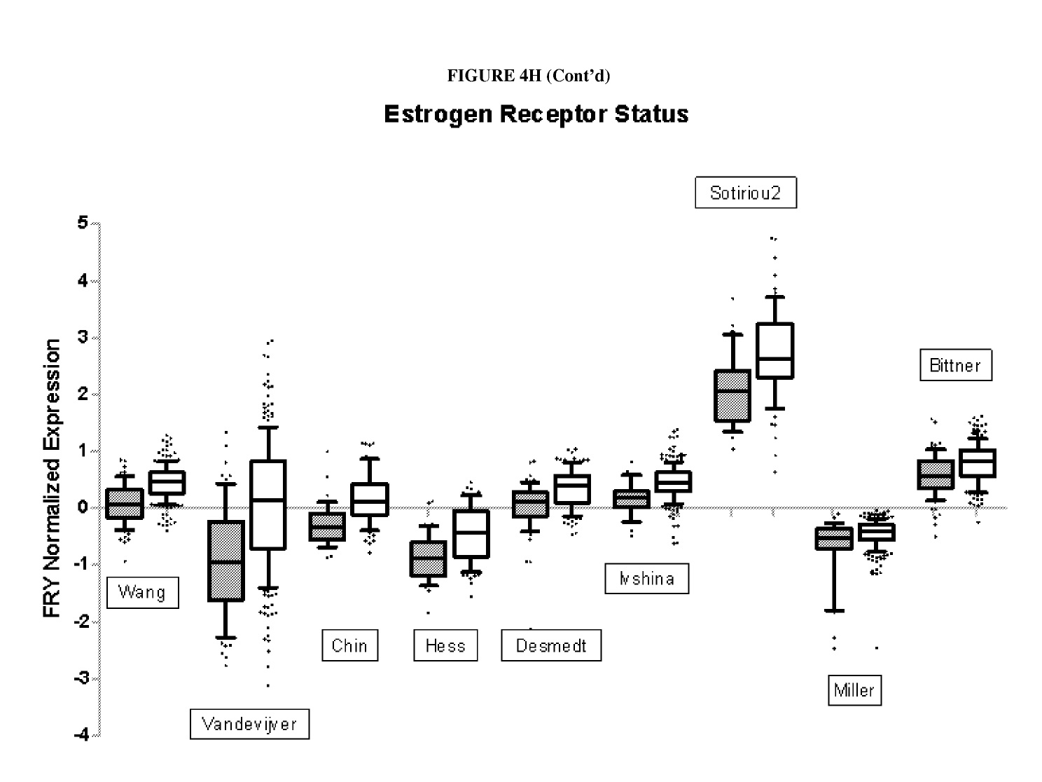

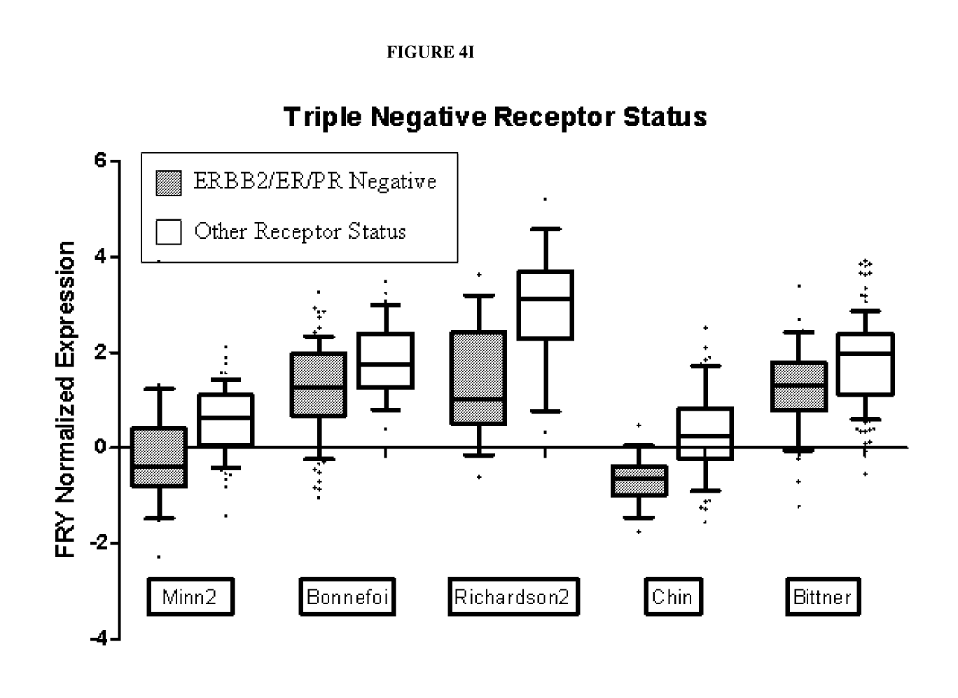

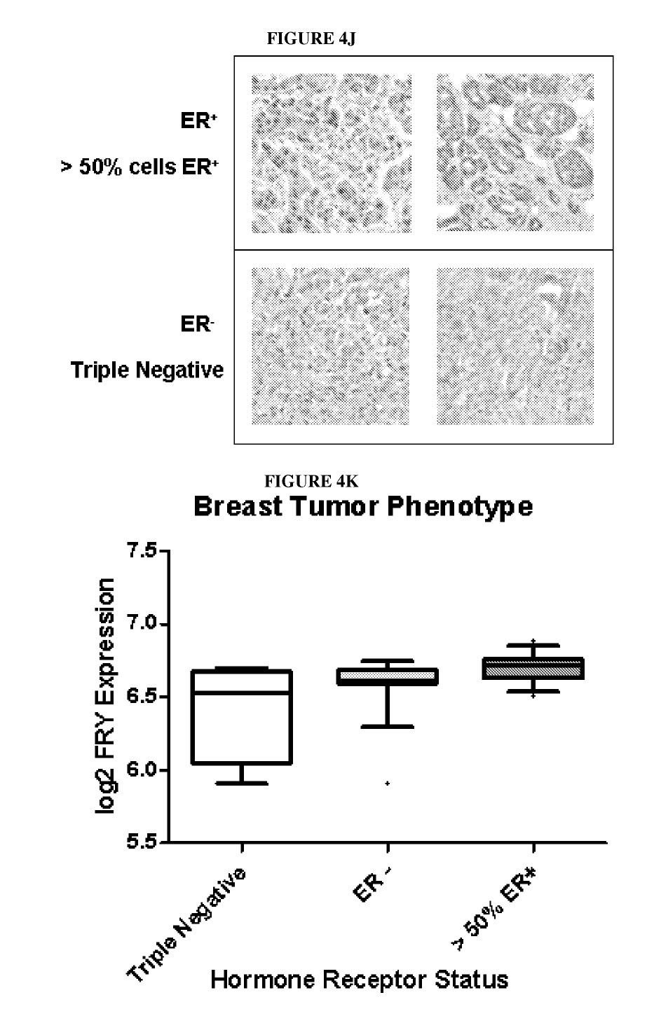

To assess the relevance of altered FRY gene expression in the clinical progression of human breast cancer, data available in the Oncomine 3.0 Cancer Profiling Database (http://www.oncomine.org) were utilized. The analysis of the study indicated that FRY expression was significantly reduced in human breast cancers compared to normal mammary tissue (p<0.0001) (FIG. 4a). This observation was confirmed at the protein level by designing and validating an anti-FRY antibody to SEQ ID NO. 3, against a peptide sequence conserved in the human and rat proteins. The antibody was then used for immunohistochemical staining and semi-quantitative image analysis of commercial breast tumor and normal tissue microarrays (U.S. Biomax, Inc.). The level of FRY expression was independently evaluated by a pathologist. Both analyses confirmed that FRY polypeptide expression was significantly reduced in tumors relative to normal mammary cells, thus validating the mRNA observations in an independent cohort.

Immunohistochemistry also revealed a significant difference in nuclear FRY polypeptide expression between tumor and normal tissue types (p<0.02) (FIG. 4 b,c). Importantly, the analysis indicated that while the nuclei of >70% of normal breast cells stained positive for FRY, only 5% of tumors analyzed showed this degree of nuclear staining (FIG. 4d). These findings indicated that loss of nuclear FRY polypeptide expression is highly correlated with the clinical phenotype of human breast cancer.

### Example 6

### Fry is Decreased in High Grade, Hormone Receptor Negative Breast Cancers

The analysis of FRY expression in ten breast cancer cohorts revealed that FRY expression was further decreased in poorly differentiated relative to well-differentiated breast tumors (n=1,860; p<0.0001) (FIG. 4e; Table 5a and b). These observations were validated at the protein level in an independent cohort using quantitative image analysis of immunohistochemically stained TMAs (N=69; p<0.02) (FIG. 4 f,g).

TABLE 5a
Tumor Grade Clinical Breast Cancer Cohorts - FRY mRNA

| | | | | | Statistical |
|---|---|---|---|---|---|
| | | | | | Analysis |
| | | | N: Sample₁ | | (KW or OA) |
| Authors | Year | Oncomine ID | Distribution | N2 | p-value3 |
| Vantveer et al. | 2002 | Vantveer_Breast | Grade 1: 12, Grade | 117 | <0.0001 (OA) |
| | | | 2: 27, Grade 3: 78 | | |
| Sotiriou et al. | 2003 | Sotiriou2_Breast | Grade 1: 16, Grade, | 98 | 0.0001 (KW) |
| | | | 2: 37, Grade 3: 45 | | |
| Miller et al. | 2005 | Miller_Breast | Grade 1: 67, Grade | 248 | <0.0001 (KW) |
| | | | 2: 128, Grade 3: 53 | | |
| Bittner et al. | 2005 | Bittner_Breast | Grade 1: 30, Grade | 278 | <0.0001 (OA) |
| | | | 2: 107, Grade 3: 141 | | |
| Ivshina et al. | 2006 | Ivshina_Breast | Grade 1: 68, Grade | 289 | <0.0001 (KW) |
| | | | 2: 166, Grade 3: 55 | | |
| Sotiriou et al. | 2006 | Sotiriou3_Breast | Grade 1: 67, Grade | 172 | <0.0001 (KW) |
| | | | 2: 46, Grade 3: 59 | | |
| Hess et al. | 2006 | Hess_Breast | Grade 1: 2, Grade 2: | 133 | <0.0001 (KW) |
| | | | 54, Grade 3: 77 | | |
| Desmedt et al. | 2007 | Desmedt_Breast | Grade 1: 30, Grade | 196 | <0.0001 (KW) |
| | | | 2: 83, Grade 3: 83 | | |
| Schmidt et al. | 2008 | Schmidt_Breast | Grade 1: 29, Grade | 200 | <0.0001 (KW) |
| | | | 2: 136, Grade 3: 35 | | |
| Finak et al. | 2008 | Finak_Breast | Grade 1: 3, Grade 2: | 53 | 0.0081 (KW) |
| | | | 23, Grade 3: 27 | | |
| Lu et al.⁴ | 2008 | Lu_Breast | Grade 1: 27, Grade | 129 | <0.0001 (KW) |
| | | | 2: 32, Grade 3: 70 | | |

TABLE 5b
Tumor Grade Clinical Breast Cancer Cohorts - FRY polypeptide

| | | | Statistical |
|---|---|---|---|
| | | | Analysis |
| Biomax Array | N: Sample | | (KW or OA) |
| ID | Distributionl | N² | p-value3 |
| BR208 | Grade 1: 22, Grade | 69 | <0.02 (OA) |
| | 2: 27, Grade 3: 20 | | |

Cohorts utilized for analysis of FRY expression in breast tumors. ¹Unless otherwise noted these samples were graded using the Elston grading system.²Sum of Grade 1, 2 and 3 samples.³The datasets being compared were tested for normality using the D'Agostino & Pearson omnibus normality test. OA: If the datasets passed the normality test a one-way ANOVA (parametric) was utilized to determine whether there was a trend in decreasing FRY expression with increasing tumor grade among patient samples. KW: If the datasets did not pass the normality test, the Kruskal-Wallis test (non-parametric) was utilized to determine whether there was a trend in decreasing FRY expression with increasing tumor grade among patient samples.⁴These samples were graded using the Richardson-Bloom grading system.

Cohorts within the Oncomine database that included annotation with respect to estrogen receptor status were identified. Analysis of these studies indicated that FRY was significantly decreased in estrogen receptor negative (ER−) breast cancers relative to estrogen receptor positive (ER+) breast cancers (n=2,555; p<0.01) (FIG. 4h; Table 6a and b). FRY was also significantly decreased in triple negative (ERBB2/ER/PR−) mammary cancers relative to cancers with other receptor statuses (n=475; p<0.01) (FIG. 4i; Table 7). The data was confirmed at the protein level in an independent cohort and demonstrated that FRY polypeptide expression was decreased in ER− breast cancers (N=16) and in triple negative cancers (N=4), relative to ER⁺ cancers with more than 50% of ER⁺ cells (N=12) (p<0.04) (FIG. 4 j,k). Reduced expression of FRY polypeptide in a limited set of ER⁻ (N=16) and ER⁺ (N=18) breast cancers also approached statistical significance (p=0.057). The analysis of breast cancer cohorts indicated that decreased FRY is highly associated with poorly differentiated, hormone receptor negative breast cancer phenotypes, which tend to be the most aggressive and malignant breast tumor types and have poor clinical outcomes.

TABLE 6a
Hormone Receptor Status Clinical Breast Cancer Cohorts - FR Y mRNA

| | | | | | Statistical |
|---|---|---|---|---|---|
| | | | | | Analysis |
| | | | N: Sample | | (ST or MW) |
| Authors | Year | Oncomine ID | Distribution | N₁ | p-value2 |
| Hedenfalk et al. | 2001 | Hedenfalk Breast | ER− 11, ER+ 10 | 21 | 0.007 (ST) |
| Gruvberger et al. | 2001 | Gruvberger Breast | ER− 30, ER+ 28 | 58 | 0.0242 (ST) |
| VandeVijver et al. | 2002 | Vandevijver Breast | ER− 69, ER+ 226 | 295 | <0.0001 (ST) |
| Sotiriou et al. | 2003 | Sotiriou2 Breast | ER− 33, ER+ 65 | 98 | 0.0001 (ST) |
| Huang et al. | 2003 | Huang Breast | ER− 15, ER+ 73 | 88 | 0.0409 (ST) |
| Hedenfalk et al. | 2003 | Hedenfalk2 Breast | ER− 6, ER+ 10 | 16 | 0.003 (MW) |
| Wang et al. | 2005 | Wang Breast | ER− 77, ER+ 209 | 286 | <0.0001 (ST) |
| Bittner et al. | 2005 | Bittner Breast | ER− 76, ER+ 149 | 225 | <0.0001 (ST) |
| Miller et al. | 2005 | Miller Breast | ER− 34, ER+ 213 | 247 | 0.007 (MW) |
| Minn et al. | 2005 | Minn2 Breast | ER− 42, ER+ 57 | 99 | <0.0001 (MW) |
| Bild et al. | 2006 | Bild Breast | ER− 48, ER+ 110 | 158 | 0.0016 (ST) |
| Ginestier et al. | 2006 | Ginestier Breast | ER− 28, ER+ 27 | 55 | 0.0003 (ST) |
| Chin et al. | 2006 | Chin Breast | ER− 43, ER+ 75 | 118 | <0.0001 (MW) |
| Ivshina et al. | 2006 | Ivshina Breast | ER− 33, ER+ 212 | 245 | <0.0001 (MW) |
| Richardson et al. | 2006 | Richardson2 Breast | ER− 24, ER+ 15 | 39 | 0.0005 (ST) |
| Hess et al. | 2006 | Hess Breast | ER− 51, ER+ 82 | 133 | <0.0001 (MW) |
| Desmedt et al. | 2007 | Desmedt Breast | ER− 64, ER+ 134 | 198 | <0.0001 (MW) |
| Lu et al. | 2008 | Lu Breast | ER− 53, ER+ 76 | 129 | <0.0001 (MW) |
| Boersma et al. | 2008 | Boersma Breast | ER− 26, ER+ 21 | 47 | 0.0021 (ST) |

TABLE 6b
Hormone Receptor Status Clinical Breast Cancer
Cohorts - FRY polypeptide

| | | | Statistical |
|---|---|---|---|
| | | | Analysis |
| Biomax, Inc | N: Sample | | (ST or MW) |
| Array ID | Distribution | N₁ | p-value₂ |
| BR962 | ER− 16, ER+ 12 | 28 | <0.03 (MW) |
| BR962 | Triple Negative: 4, | 16 | <0.04 (MW) |
| | ER+ 12 | | |

Cohorts utilized for analysis of FRY expression in breast tumors. ¹ Sum of estrogen receptor positive and estrogen receptor negative samples.² The datasets being compared were tested for normality using the D'Agostino & Pearson omnibus normality test. ST: If the datasets passed the normality test the Student's t-test (parametric) was utilized to determine whether the mean FRY expression in estrogen receptor negative samples is significantly different and less than that in estrogen receptor positive samples with. MW: If the datasets did not pass the normality test, the Mann-Whitney test (non-parametric) was utilized to determine whether the mean FRY expression in ER negative samples is significantly lower than that in ER positive samples.

TABLE 7
Triple Negative Clinical Breast Cancer Cohorts - FRY mRNA

| | | | | | Statistical |
|---|---|---|---|---|---|
| | | | | | Analysis |
| | | | | | (ST or MW) |
| Authors | Year | Oncomine ID | N: Sample Distribution | N¹ | p-value₂ |
| Bittner et al. | 2005 | Bittner_Breast | Triple Negative: 39, Other | 160 | 0.0019 (ST) |
| | | | Receptor Status: 121 | | |
| Minn et al. | 2005 | Minn2_Breast | Triple Negative: 25, Other | 88 | 0.0004 (MW) |
| | | | Receptor Status: 63 | | |
| Chin et al. | 2006 | Chin_Breast | Triple Negative: 19, Other | 78 | <0.0001 (ST) |
| | | | Receptor Status: 59 | | |
| Richardson et | 2006 | Richardson2 Breast | Triple Negative: 18, Other | 37 | 0.0003 (ST) |
| al. | | | Receptor Status: 19 | | |
| Bonnefoi et | 2007 | Bonnefoi Breast | Triple Negative: 80, Other | 112 | 0.0026 (ST) |
| al. | | | Receptor Status: 32 | | |

Oncomine cohorts utilized for FRY analysis in breast tumors. ¹ Sum of triple negative samples and samples with other receptor statuses.² The datasets being compared were tested for normality using the D'Agostino & Pearson omnibus normality test. ST: If the datasets passed the normality test the Student's t-test (parametric) was utilized to determine whether the mean FRY expression in triple negative samples is significantly different and less than that in samples with other receptor statuses. MW: If the datasets did not pass the normality test, the Mann-Whitney test (non-parametric) was utilized to determine whether the mean FRY expression in triple negative samples is less than that in samples with other receptor statuses between these groups is significantly different.

### Example 7

### FRY Plays a Role in Epithelial Cell Differentiation and Development

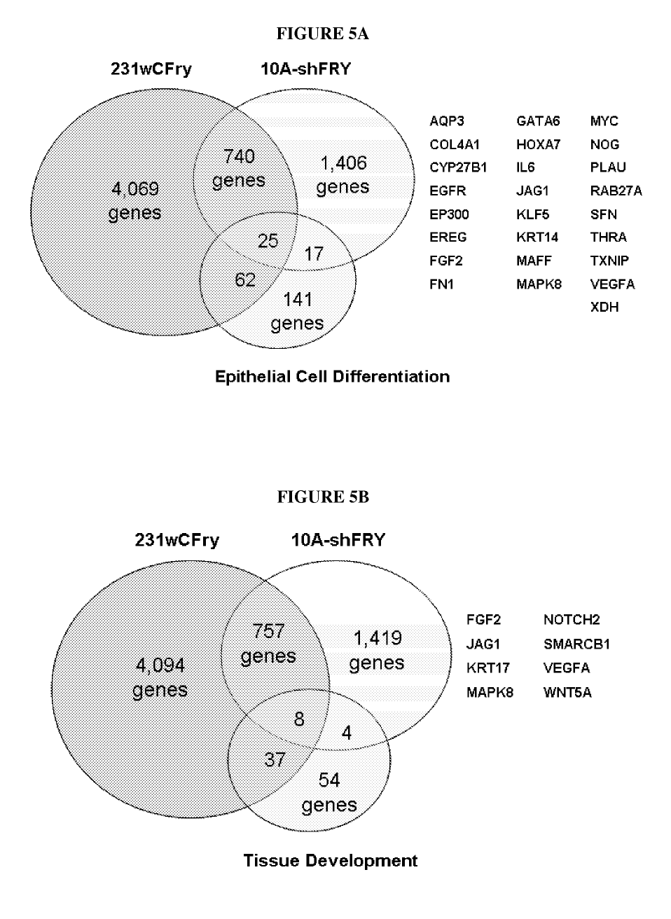

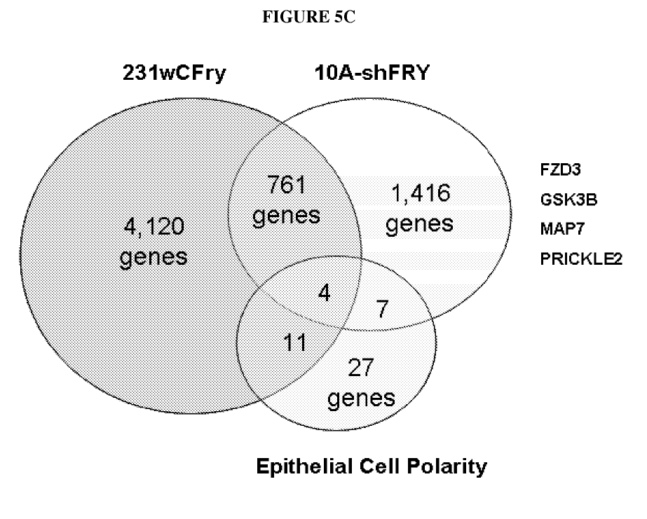

The in-vitro and in-vivo analyses of the isogenic pairs of cell lines (MDA-MB-231 and 231wCFRY; MCF10A and 10A-shFRY) implicated FRY in epithelial cell differentiation, adhesion and mobility. Gene expression profiling in-silico analysis using of IPA (Ingenuity® Systems, www.ingenuity.com) confirmed a similar role for FRY in gene networks related to maintenance of epithelial cell architecture, differentiation, motility, cell-to-cell signaling and cell adhesion (Table 8). Altered FRY levels significantly changed the expression levels in 42% (104/245) of the genes implicated in epithelial cell differentiation, 48% (49/103) of genes involved in tissue development, and 45% (22/49) of genes involved in cell polarity (FIG. 5). Ingenuity® Pathway Analysis software was utilized for functional analysis of genes which were commonly significantly (p<0.05; 765 genes, Table 5 (a) and (b)) altered in both pairs of isogenic cell lines (MDA-MB-231/231wCFry and 10A-CV/10A-shFRY). (a, b, c) FRY-responsive genes elucidated in epithelial cell differentiation, tissue development and cell polarity. Genes listed to the right of each Venn diagram were commonly altered in both pairs of isogenic cell lines (FIG. 5). The canonical pathway most affected by ectopic FRY expression in tumor cells or decreased FRY expression in normal mammary epithelial cells was the Wnt/β-Catenin signaling pathway (Table 9), which plays a crucial role in maintaining epithelial cell polarity. Decreased FRY expression in 10A-shFRY reduced β-Catenin expression and ectopic expression of FRY in the 231wCFRY cell line restored β-Catenin expression (FIG. 6a).

TABLE 8
FRY Responsive Genes.

ABCC3
ABHD15
ACAD8
ACAT2
ACTR1A
ADAM8
ADAM17
ADAMTS15
ADD3
AFAP1L1
AFF4
AGR2
AIG1
AKR1C3
AKR1C1/AKR1C2
ALAS2
ALCAM
ALDH1L2
ALS2CL
AMOTL1
ANGPTL4
ANK3
ANKRD28
ANKRD50
ANXA8L2
AOX1
AP1S3
APAF1
APBB2
AQP3
AREG/AREGB
ARF3
ARHGAP32
ARHGEF9
ARID4A
ARL4D
ARNTL2
ARRB2
ARSD
ARV1
ASAP1
ASNS
ATF3
ATP2A2
ATP5G3
ATP9A
AXL
B3GNT5
B9D1
BAMBI
BBS7
BRI3BP
BTG1
BTG3
C10orf47
C11orf1
C11orf75
C12orf39
C13orf18
C15orf48
C16orf52
C16orf53
C16orf74
C17orf97
C18orf25
C19orf28
C1orf21
C1orf53
C1S
C21orf7
C2orf74
C3orf21
C4orf21
C5orf46
C6orf52
C6orf132
C6orf168
C7orf10
C8orf55
C9orf3
C9orf150
C9orf123
CA12
CABYR
CALHM2
CALM1
CAMTA1
CAMTA2
CAPRIN2
CARD8
CARS
CASP6
CBVWD1
CCDC123
CCDC90A
CCL20
CCNA1
CCNA2
CD24
CD44
CD2BP2
CDC14B
CDC42EP1
CDCP1
CDH4
CDH18
CDIPT
CDK2
CDK12
CDV3
CFB
CFLAR
CHD2
CHD9
CHIC1
CHST11
CIAO1
CITED2
CLDN4
CLEC2B
CLGN
CLK4
CLMN
CLNS1A
CLSPN
CLTA
CMTM6
CNIH4
CNST
COBLL1
COG3
COL12A1
COL17A1
COL4A1
COMMD4
CPD
CPPED1
CRNDE
CSNK1E
CSNK2A1
CTSB
CTSC
CUL1
CXCL1
CXCL2
CXCL3
CXXC5
CYLD
CYP1B1
CYP24A1
CYP27B1
DAZAP2
DCTN1
DDAH1
DDX5
DDX17
DDX60
DEM1
DERL3
DFNA5
DHFRL1
DIDO1
DIEXF
DIP2C
DIS3
DIS3L2
DMRT1
DNAJC21
DSC2
DSE
DST
DUSP1
DUSP4
DUSP5
DUSP6
DUSP10
DUSP16
DYNC1H1
ECHDC3
EDEM2
EDEM3
EDIL3
EFNA5
EFTUD1
EGFR
EHF
EID2
EID2B
EIF2S3
EIF4E9
EIF4EBP1
EIF4EBP2
ELK3
ELL2
EMB
EMP1
ENPP1
ENY2
EP300
EPAS1
EPHA2
EPHA4
EPHB2
EPM2AIP1
ERCC8
EREG
ERMP1
ESYT2
ETFB
EVI5
F3
FAIM3
FAM107B
FAM133B
FAM162A
FAM167A
FAM175A
FAM201A
FAM20C
FAM24B
FAM27E3
FAM63B
FAM69A
FAM83A
FARP1
FBLIM1
FBXL5
FBXO11
FERMT1
FGD6
FGF2
FGF11
FGF13
FGFR1
FKBP15
FLJ10120
FLRT2
FLRT3
FMNL2
FN1
FOSL2
FRG1
FRMD6
FSIP1
FST
FSTL1
FSTL5
FXC1
FXYD3
FZD3
FZD7
GALNT6
GATA6
GBP1
GCNT1
GCOM1
GDAP1
GEMIN6
GIT2
GJB3
GK
GLS
GLUD2
GNA15
GNAI1
GNPAT
GOLGB1
GOPC
GOSR2
GPCPD1
GPNMB
GPR87
GPR110
GPR137C
GPRC5B
GPX7
GRIK2
GSK3B
GTF2H5
GYG1
H2AFV
HBEGF
HDAC9
HERPUD1
HFE
HIST1H2BD
HK2
HOXA7
HOXC9
HOXC11
HOXC13
HP1BP3
HPGD
HRCT1
HRH1
HS3ST3B1
HSPA2
HSPA13
HTATIP2
HTRA1
ICAM1
IER5L
IFT27
IGFBP3
IL6
IL32
IL13RA1
IL13RA2
IL1RL1
IL31RA
IL6ST
ING5
INHBA
INHBE
INSIG1
INTS6
IPO9
IQCB1
IRS1
IRX5
ISL2
ITGA3
ITGB3
ITGB4
ITGB8
ITPR2
JAG1
JHDM1D
JMJD1C
JRK
JUB
KANK1
KCNJ15
KCNK1
KCNMA1
KHDRBS3
KIAA0391
KIAA0564
KIAA1217
KIAA1279
KIAA1310
KIAA1430
KIAA1644
KIAA1797
KIAA0664L3
KIAA1324L
KIF7
KLF5
KLF7
KLHL29
KLK6
KMO
KRCC1
KRT14
KRT17
KRT8L2
KYNU
LAMA3
LAMB3
LAMC1
LAMC2
LARP6
LCN2
LETM2
LMAN1
LMBR1
LMO7
LOC25845
LOC81691
LOC221710
LOC283788
LOC284561
LOC339290
LOC389834
LOC651250
LOC728855
LOC100129637
LOC100292680
LOC100499467
LOC100505761
LOC100131199
LOX
LRRC16A
LUM
LYRM2
M6PR
MAFB
MAFF
MALL
MAN1A1
MAN2C1
MANSC1
MAOA
MAP7
MAP3K3
MAP4K4
MAPK8
MARCKS
MAST4
MBD1
MBP
MCPH1
MCTP1
MDM4
MED13L
MFAP3L
MIA
MICAL2
MICAL3
MIPOL1
MLLT4
MMP14
MON1B
MPP5
MPZL3
MRPS21
MSI2
MTHFD2
MTHFD1L
MTL5
MTSS1
MXD1
MYC
MYO10
MYO1E
MYPN
NAMPT
NASP
NCOR1
NCRNA00292
NDUFB2
NEDD4L
NEGR1
NEK9
NETO2
NF2
NFX1
NHEDC2
NLN
NMD3
NOG
NOTCH2
NPEPL1
NPY1R
NR1D2
NR2F2
NR3C1
NRIP1
NRP2
NUDT4
NUDT21
NUPR1
OAZ3
OCLN
OGFRL1
OPA3
OSBPL6
OTUD4
P2RY2
PAICS
PAPOLA
PAPSS2
PAQR5
PAWR
PAX8
PBXIP1
PCDH7
PCGF2
PCGF6
PCK2
PCM1
PDE4D
PDE4DIP
PDGFC
PDK4
PDLIM4
PDLIM5
PDXK
PDZD2
PDZK1IP1
PEG10
PGBD1
PHF14
PHLDB2
PIGC
PIM1
PIR
PLA2G4A
PLAC2
PLAU
PLAUR
PLSCR1
PLXNA1
POLE4
POLR2C
POMT1
PON2
POSTN
POU2F2
PPAP2B
PPM1A
PPP1R3C
PPP2CA
PPP2R5D
PPP3CC
PRICKLE1
PRICKLE2
PRKAG2
PRKAR2B
PRNP
PROCR
PROM2
PRRC2C
PSAT1
PSCA
PSTPIP2
PTER
PTGS2
PTPN12
PTPN14
PTPN18
PTPRE
PTPRJ
PTPRM
PVRL2
PWWP2B
QKI
QPCT
QRSL1
RAB31
RAB27A
RAB4A
RAGE
RANBP17
RAP2B
RASSF9
RB1CC1
RBBP6
RBM8A
RBMS3
RBPMS
RCAN2
RCOR1
RFC1
RGMB
RGNEF
RHOB
RHOBTB1
RHOF
RHOQ
RIMS2
RIPK2
RIT1
RLIM
RNF13
RNF146
RNF144B
RNFT2
ROR1
RSL24D1
RSPRY1
RTTN
SAA1
SAMD5
SBF2
SBNO1
SCD
SDC2
SDR16C5
SEC62
SEC24A
SELENBP1
SEMA3C
SEMA3D
SEMA3F
SEMA5A
SEMA6A
SEMA7A
SERPINA1
SERPINA3
SERPINE1
SESN2
SETD5
SETD7
SFN
SGCB
SGMS2
SH3RF3
SHISA2
SIK3
SIM2
SIPA1L1
SIPA1L2
SIRPA
SIRT3
SKA2
SKIV2L2
SLAIN1
SLC16A6
SLC16A14
SLC1A1
SLC22A3
SLC25A17
SLC25A29
SLC25A37
SLC29A1
SLC35B4
SLC39A8
SLC44A2
SLC44A3
SLC45A4
SLC4A11
SLC6A8
SLC6A14
SLC6A15
SLC7A2
SLFN5
SLITRK5
SLITRK6
SLK
SMAD2
SMARCB1
SMEK2
SMOX
SMTN
SMURF2
SNAPC3
SNHG5
SNRK
SNRPE
SOD2
SORBS1
SOX5
SP140/SP140L
SPA17
SPIN3
SPOCK1
SPTLC3
SQRDL
SREK1
SSH1
SSU72
STC1
STEAP1
STK38
STX6
STX16
SUGT1
SULT1A1
SULT1A2
SULT1A3/SULT1A4
SUN2
SYBU
SYNGR1
SYNM
TAB2
TAB3
TAF11
TANC2
TAP2
TATDN3
TBL1XR1
TBX3
TCN1
TCP11L1
TET2
TFCP2L1
TFPI
TFPT
TGIF1
THADA
THRA
TIGD2
TLK2
TLR2
TLR4
TMCC3
TMCO6
TMEFF1
TMEM2
TMEM19
TMEM139
TMEM154
TMEM63C
TNFAIP3
TNFAIP6
TNFAIP8
TNFRSF10D
TNFRSF11A
TNIP1
TNIP3
TPM1
TPM2
TRAF3IP2
TRAK1
TRAM1L1
TRIB1
TRIB3
TRIM2
TRIM8
TRIM23
TRIM29
TRIOBP
TRPS1
TSPAN13
TTC9
TTC14
TTC22
TUBE1
TUSC3
TWIST1
TWSG1
TXNIP
TXNL4B
TYSND1
UBAP2L
UBE2H
ULK3
UNC5C
UQCRB
USP25
USP46
USP53
VAMP4
VANGL1
VEGFA
VEZT
VNN1
VRK3
VSIG10L
VWA5A
WISP3
WNT5A
WWC3
XBP1
XDH
ZADH2
ZBED6
ZBTB10
ZBTB44
ZCCHC2
ZCCHC7
ZDHHC2
ZDHHC3
ZDHHC17
ZEB2
ZFP36L1
ZFYVE21
ZKSCAN1
ZNF22
ZNF117
ZNF131
ZNF174
ZNF232
ZNF236
ZNF302
ZNF346
ZNF365
ZNF451
ZNF551
ZNF558
ZNF785
ZNF789
ZNF264/ZNF805
ZNRF1

TABLE 9
IPA Canonical Signaling Pathways Represented by FRY Responsive Genes.
Top Canonical Pathways

| | |
|---|---|
| ILK Signaling | Nicotinate and Nicotinamide Metabolism |
| Antiproliferative Role of TOB in T Cell Signaling | TGF-0 Signaling |
| Colorectal Cancer Metastasis Signaling | Aryl Hydrocarbon Receptor Signaling |
| Axonal Guidance Signaling | Bladder Cancer Signaling |
| Hepatic Fibrosis/Hepatic Stellate Cell Activation | Sonic Hedgehog Signaling |
| Clathrin-mediated Endocytosis Signaling | Dopamine Receptor Signaling |
| Glucocorticoid Receptor Signaling | Huntington's Disease Signaling |
| Coagulation System | Role of Tissue Factor in Cancer |
| RAR Activation | Estrogen Receptor Signaling |
| NF-xB Signaling | p53 Signaling |
| VDR/RXR Activation | ERK/MAPK Signaling |
| PXR/RXR Activation | Acute Phase Response Signaling |
| Glioblastoma Multiforme Signaling | PPAR Signaling |
| Glioma Invasiveness Signaling | Riboflavin Metabolism |
| Sulfur Metabolism | Wnt/β-catenin Signaling |
| D-glutamine and D-glutamate Metabolism | Sphingolipid Metabolism |
| IL-8 Signaling | MIF-mediated Glucocorticoid Regulation |
| IL-17A Signaling in Airway Cells | Ephrin Receptor Signaling |
| Cyclins and Cell Cycle Regulation | Production of Nitric Oxide and Reactive Oxygen |
| | Species in Macrophages |
| Nitrogen Metabolism | Chondroitin Sulfate Biosynthesis |
| Role of IL-17A in Psoriasis | Toll-like Receptor Signaling |
| Tumoricidal Function of Hepatic Natural Killer | Cysteine Metabolism |
| Cells | |
| Molecular Mechanisms of Cancer | Cholecystokinin/Gastrin-mediated Signaling |
| Ovarian Cancer Signaling | Cell Cycle Regulation by BTG Family Proteins |
| IL-17A Signaling in Gastric Cells | IL-17A Signaling in Fibroblasts |

Use of the isogenic cell lines indicated that FRY also plays a role in cell-cell adhesion and mobility. The expression of α4-Integrin (ITGA4), an important cell adhesion molecule (CAM) involved in cellular migration is also a receptor for fibronectin, a molecule that plays major roles in cell adhesion, growth, migration and differentiation. Western blot analysis illustrated that decreased FRY expression in 10A-shFRY reduced ITGA4 expression and ectopic expression of FRY in the 231wCFRY cell line restored ITGA4 expression (FIG. 6b). FIG. 6b illustrates that when FRY expression is decreased (10A-shFRY) α4-Integrin is decreased and when ectopic Fry is expressed (231wCFry), α4-Integrin is increased. Quantification by normalization to β-actin protein expression (N=3); Protein expression levels are presented as % of β-actin.

### Example 8

### Fry is Decreased in Prostate, Ovarian, Lung, Brain and Blood Cancers

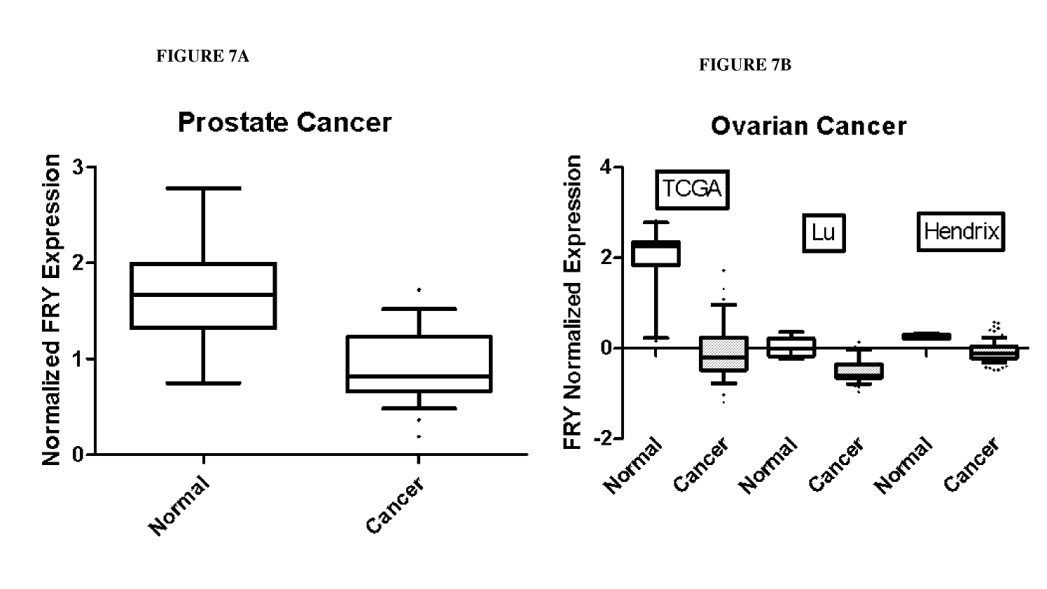

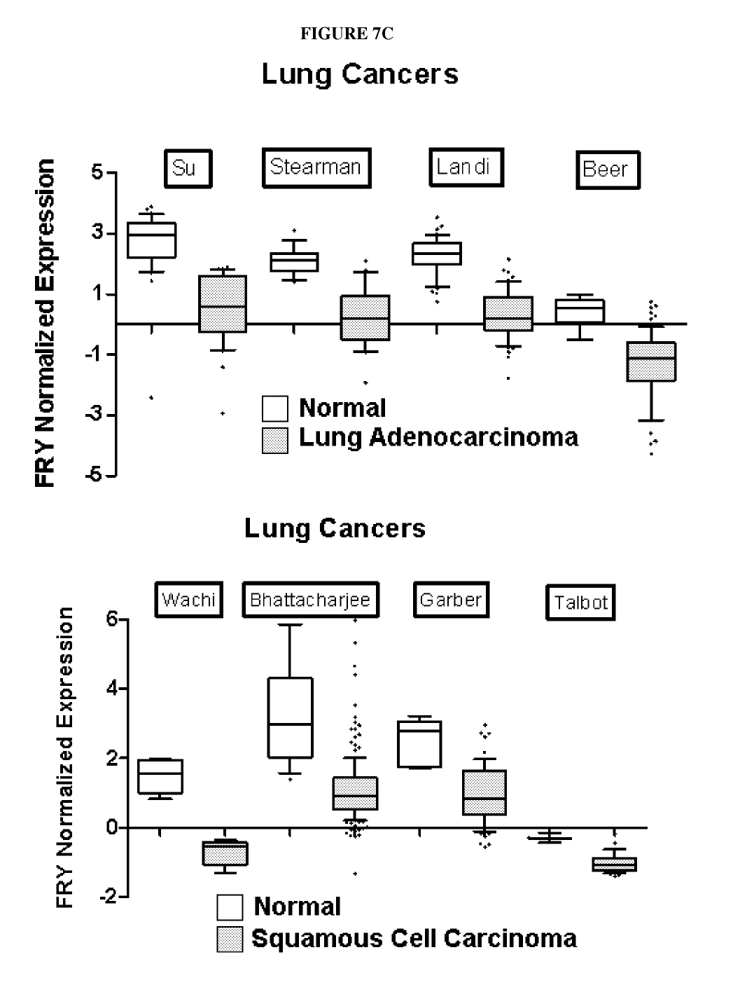

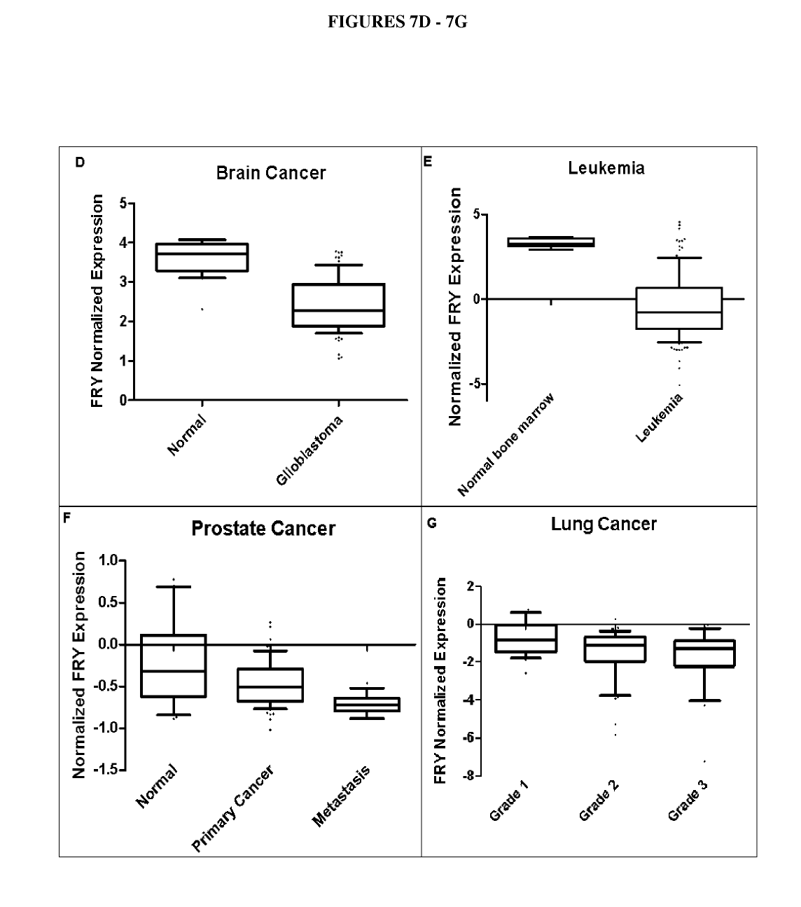

Data available in the Oncomine 3.0 Cancer Profiling Database was used to identify clinically annotated prostate, ovarian, lung, brain and blood cancer cohorts. The analysis of prostate cancer cohorts (p<0.003) (Normal: 32; Cancers: 114), ovarian cancer cohorts (p<0.008) (Normal: 19; Cancers: 182) and lung cancer cohorts (p<0.008) (Normal: 136; Cancers: 452) all indicated that FRY was significantly decreased in carcinomas relative to normal tissues (FIG. 7 a,b,c). FRY expression was decreased in glioblastomas relative to normal brain tissue (p<0.0001) (Normal: 23; Cancers: 81) and that FRY was decreased in leukemia cases relative to normal bone marrow (p<0.0002) (Normal: 6; Cancers: 121) (FIG. 7 d,e).

The expression of FRY was lowest in prostate cancers which metastasized compared to prostate cancers which did not metastasize (Primary Cancer: 65; Metastasis Present: 24) (p<0.0001; FIG. 7) and that, consistent with our observations in human breast cancer, FRY expression was significantly lower in high grade, poorly differentiated lung cancers relative to its expression in well-differentiated lung carcinomas (p<0.03; FIG. 7 f,g).

### Example 9

### SNAP Analysis

SNAP (Screening for Non-Acceptable Polymorphisms) predicts the functional consequences of single amino acid substitutions in both binary (neutral/non-neutral, with respect to wild type function) and scored form (−100 to +100, where negative predictions are neutral, positive are non-neutral, and higher absolute values of scores indicate higher reliability of the binary prediction) (Bromberg and Rost 2007). SNAP also provides the likely functional (as opposed to structural) importance of each amino acid in the protein sequence by computing the SNAP-BLOSUMB62 score—for each wild-type residue, the average SNAP score of substitutions allowed by the BLOSUM62-matrix at cutoff ≧0. As with regular SNAP scores, SNAP-BLOSUM62 scores ≦0 indicate that a specific sequence position is not likely functionally significant, and a score >0 indicates that this location is probably functionally significant. The human FRY polypeptide sequence was taken from the USCS Genome Browser (Q5TBA9; FRY_HUMAN). The SNAP score (−48) for an amino acid change at codon 661 (where the F344 rat had an Aspartic acid to Glutamic acid mutation) indicated that this mutation is not likely to cause functional consequences. The BLOSUM62 score (−19) of position 661, additionally suggested that it may not be functionally significant. The SNAP scores for all potential substitutions at human codon 2171 which coincided with the mutation at codon 2170 observed in the Fisher F344 rat strain were analyzed. The SNAP score for an amino acid substitution at codon 2171 (where the F344 rat had an Alanine to Serine mutation; score+15) indicated that an amino acid change at this location is likely to have functional consequences. The SNAP-BLOSUM62 score indicated that this location is functionally significant (score+8).

### Example 10

### Fry Protein Expression is Lower in Malignant Breast Lesions than in Benign Lesions

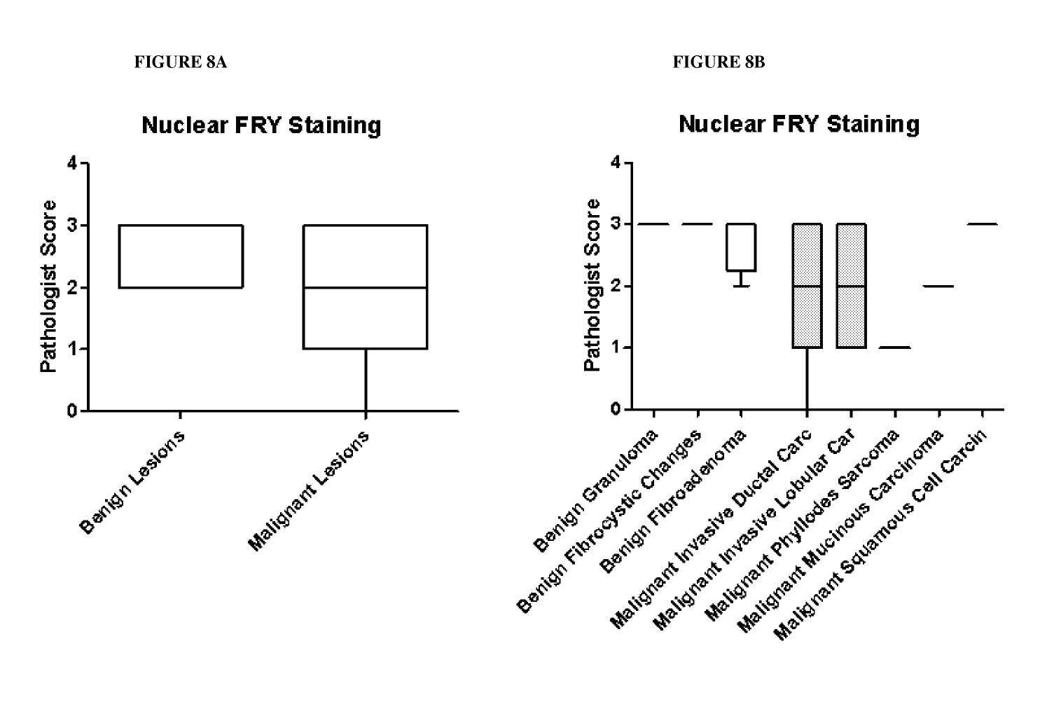

Based on pathologist scores, nuclear FRY protein expression was significantly higher in benign breast lesions (fibroadenoma, granuloma and breast tissue with benign fibrocystic changes) compared to malignant lesions (invasive ductal carcinoma, invasive lobular carcinoma, phyllodes sarcoma, mucinous carcinoma and squamous carcinoma) based on analysis by the nonparametric, Mann Whitney Test (p<0.025). FIG. 8 illustrates nuclear FRY protein expression was significantly higher in benign breast lesions compared to malignant lesions. (a) and (b) are floating bar charts designating min to max for each group with a line at the median. Pathologist scores are from 0-3 (0: <10% of epithelial cell nuclei were positive for FRY, 1: 10-40% of epithelial cell nuclei stained positive for FRY, 2: 40-70% of epithelial cell nuclei stained positive for FRY, 3: 70-100% of epithelial cell nuclei stained positive for FRY.

The specification is most thoroughly understood in light of the teachings of the references cited within the specification. The embodiments within the specification provide an illustration of embodiments of the invention and should not be construed to limit the scope of the invention. The skilled artisan readily recognizes that many other embodiments are encompassed by the invention. All publications U.S. patents and GenBank sequences cited in this disclosure are incorporated by reference in their entireties. The citation of any references herein is not an admission that such references are prior art to the present invention.

Those skilled in the art will recognize, or be able to ascertain using no more than routine experimentation, many equivalents to the specific embodiments of the invention described herein. Such equivalents are intended to be encompassed by the following embodiments.

1. A method for diagnosing cancer in a subject comprising:
a. detecting a level of a FRY polypeptide or a polynucleotide encoding a FRY polypeptide in a biological sample from the subject; and
b. comparing the level detected in the subject's sample to a standard level in a corresponding non-cancerous sample; and
c. diagnosing cancer when the level in the sample from the subject is less than the standard level.

2. The method of claim 1 wherein the level of the FRY polypeptide is determined by detecting binding of the FRY polypeptide in the sample to an antibody that specifically binds to a FRY polypeptide.

3. The method of claim 2 wherein the antibody binds an epitope comprising the peptide sequence WGVRRRSLDSLDKC (SEQ ID NO:3) within the FRY polypeptide.

4. The method of claim 2, wherein the antibody is a polyclonal antibody.

5. The method of claim 2, wherein the antibody is a monoclonal antibody.

6. The method of claim 1 wherein the level of the polynucleotide encoding the FRY polypeptide is determined by detecting the hybridization to the polynucleotide by a second polynucleotide comprising the sequence of SEQ ID NO:1, a fragment thereof the complement of SEQ ID NO:1, or a fragment thereof.

7. The method of claim 1, wherein the cancer is selected from the group consisting, of epithelial cell cancer, breast cancer, prostate cancer, ovarian cancer, lung cancer, brain cancer and blood cancer.

8. The method of claim 1, wherein the cancer cells possess a stem cell phenotype.

9. The method of claim 7, wherein the breast cancer is a hormone receptor negative cancer.

10. The method of claim 9, wherein the hormone receptor is selected from the group consisting of an estrogen receptor a progesterone receptor and human epidermal growth factor receptor 2, or any combination thereof.

11. The method of claim 9, wherein the hormone receptor negative cancer is a triple negative breast cancer.

12. (canceled)

13. (canceled)

14. (canceled)

15. A method of identifying a subject at risk of developing cancer comprising detecting a susceptibility polymorphism in the FRY gene of the subject, wherein the presence of the susceptibility polymorphism is predictive of increased risk for developing cancer.

16. The method of claim 16, wherein the cancer is selected from the group consisting of epithelial cell cancer, breast cancer, prostate cancer, ovarian cancer, lung cancer, brain cancer and blood cancer.

17. (canceled)

18. (canceled)

19. (canceled)

20. (canceled)

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. An isolated antibody that specifically binds to a FRY polypeptide.

33. The antibody of claim 32, wherein the antibody is a polyclonal antibody.

34. The antibody of claim 32, wherein the antibody is a monoclonal antibody.

35. The antibody of claim 32 wherein the antibody binds an epitope comprising the peptide sequence WGVRRRSLDSLDKC (SEQ ID NO:3) within the FRY polypeptide.

36. (canceled)

37. (canceled)

38. (canceled)

39. A kit for the diagnosis, prognosis or monitoring the treatment of a cancer comprising one or more detection reagents, wherein at least one detection reagent is the antibody of claim 32, and said kit optionally further comprises one or more reagents selected from the group consisting of reagents which specifically bind to a polynucleotide encoding a FRY polypeptide, and reagents which specifically bind to a FRY polypeptide in a biological sample from a subject.

40. The kit of claim 39 further comprising reagents for performing nucleic acid hybridization.

41. The kit of claim her comprising reagents for immunohistochemistry or for performing PCR.

42. (canceled)
